(19) United States
(12) Patent Application Publication
(10) Pub. No.: US 2024/0174703 A1
(43) Pub. Date: May 30, 2024

(54) THREE-COORDINATE AU(I) PROBES AND USE IN SELECTIVELY DISRUPTING MITOCHONDRIA IN CANCER CELLS

(71) Applicant: University of Kentucky Research Foundation, Lexington, KY (US)

(72) Inventors: Randall Tyler Mertens, Lexington, KY (US); Samuel Gorman Awuah, Lexington, KY (US)

(21) Appl. No.: 18/280,111

(22) PCT Filed: Mar. 4, 2022
(86) PCT No.: PCT/US22/18941

§ 371 Date: Sep. 1, 2023

Related U.S. Application Data
(60) Provisional application No. 63/156,576, filed on Mar. 4, 2021.

(51) Int. Cl. C07F 9/6561; A61P 35/00; C07F 1/00; C07F 9/80

(52) U.S. Cl. CPC C07F 9/6561; A61P 35/00; C07F 1/005; C07F 9/80

(57) ABSTRACT

The presently-disclosed subject matter tri-coordinate Au(I) complexes, and methods of using tri-coordinate Au(I) complexes for selectively disrupting mitochondrial structure of target cancer cells.

### RELATED APPLICATIONS

This application claims priority from U.S. Provisional Application Ser. No. 63/156,576 filed Mar. 4, 2021, the entire disclosure of which is incorporated herein by this reference.

### GOVERNMENT INTEREST

This invention was made with government support under grant number P20 GM130456 awarded by the National Institutes of Health. The government has certain rights in the invention.

### TECHNICAL FIELD

The presently-disclosed subject matter relates to a gold based approach to perturb mitochondrial structure in cancer cells with high selectivity over normal epithelial cells. The presently-disclosed subject matter also relates to tri-coordinate Au(I) complexes, and methods of using tri-coordinate Au(I) complexes for selectively disrupting mitochondrial structure of target cancer cells.

### INTRODUCTION

Mitochondria are dynamic organelles that control ATP production, biosynthesis of macromolecules, and signaling. Often referred to as the powerhouse of the cell, the mitochondria have a well-defined but complicated structure with important functional implications. The mitochondrial outer membrane of envelops the inner-membrane energy hub, which protects a dense protein rich matrix.¹ The large surface area of the inner membrane is characterized by a macromolecular folding, known as cristae.

There exists an intimate relationship between mitochondria morphology and cellular bioenergetics.² A key dynamic parameter of regulated mitochondria function is fusion and fission.³ With reference to FIG. 1, a balanced cycle of fusion and fission is critical for physiology and the loss thereof has adverse implications on cell function, leading to inflammation, ageing, neurodegeneration, cancer.⁴⁻⁵ Thus, tools to study mitochondrial structure is an unmet need and an attractive approach for developing new therapeutics.

Of interest in the context of the presently disclosed subject matter are mitochondria probes and targeting approaches, and particularly the synthetic control of mitochondrial homeostasis. Gold agents have recently been developed, which interact with the mitochondrial oxidative phosphorylation (OXPHOS) machinery and cellular metabolism. Gold compounds can be tuned for mitochondrial localization.⁶

Increasing evidence suggests that mitochondrial biological processes participate in acquired drug resistance. Specifically, loss of the mitochondrial protein caseinolytic peptidase B protein homolog (CLPB) compromises mitochondrial structural and functional integrity leading to acute myeloid leukemia (AML) sensitization to apoptosis.⁷ In this context, it is contemplated that small-molecules could disrupt mitochondrial structure to be of therapeutic benefit. However, synthetic alteration of oxidation states or ligand environment of gold compounds for direct mitochondria interaction or targeting of distinct locales is unknown.

The development of gold-based small-molecules for biological use received a major boost following the FDA approval of auranofin for the treatment of rheumatoid arthritis.⁸ Over the past decade, several gold compounds have been synthesized for potential therapeutic of a plethora of diseases.⁹⁻¹³ Their unique properties have alluded to unique behavior both in vivo and in vitro in comparison to other transition metal-based alternatives.¹⁴⁻¹⁹ Despite the breadth of complex libraries developed thus far, the unique geometry of tri-coordinated Au(I) complexes²⁰⁻²⁵ are uncommon and their therapeutic potential left untapped.²⁶⁻²⁸ Bourrisou and coworkers have demonstrated catalytic capability with tri-coordinate complexes due to their distinct reactivity.^{27, 29-32}

As disclosed herein, the effect of geometrically unique gold complexes on mitochondria morphology and functional consequences in normal or cancerous epithelial cells were explored.

As further disclosed herein, the approach included the diversification of compounds using different NAN-bidentate ligands to tune lipophilicity and monodentate arsine or phosphine ancillary ligands to define a unique degree of asymmetry for interaction with biomolecules in a manner distinct from other transition metal complexes.

As also reported herein, the structure activity relationship (SAR) enabled the selection of potent candidates that rapidly perturb the structure of mitochondria, as observed using transmission electron microscopy. The phenotype observed was sharply different from other agents of known mode of action. Additionally, immunoblotting, respirometry, and quantitative proteomics studies corroborate the discovery of unique gold compounds with specificity for mitochondrial structure.

As further reported herein, the anticancer potential of the compounds was studied and it was found that they possess an appreciable selectivity to kill cancer cells compared to normal ones. Preliminary toxicity studies in mice demonstrate that tolerance safely at 10 mg/kg, highlighting the efficacy for metal-based compounds for mitochondrial biology.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a compound that is a tri-coordinate Au(I) complexes, or a pharmaceutically acceptable salt thereof. The presently-disclosed subject matter also includes a pharmaceutical composition including tri-coordinate Au(I) complex as disclosed herein and a pharmaceutically-acceptable carrier. The presently-disclosed subject matter also includes methods of conferring anti-cancer activity to a cancer cell. The presently-disclosed subject matter also includes use of a compound disclosed herein in a medicament for the treatment of a cancer. The presently-disclosed subject matter also includes methods of modulating mitochondrial function in a cell. The presently-disclosed subject matter also includes use of a compound disclosed herein in a medicament for the treatment of a condition involving mitochondrial dysfunction. The presently-disclosed subject matter also includes methods of increasing reactive oxygen species (ROS) in a cell. The presently-disclosed subject matter also includes use of a compound disclosed herein in a medicament for increasing reactive oxygen species (ROS) in a cell.

Some embodiments of the presently-disclosed subject matter include a compound of any one of the following formulae (I)-(XII), or a pharmaceutically-acceptable salt thereof

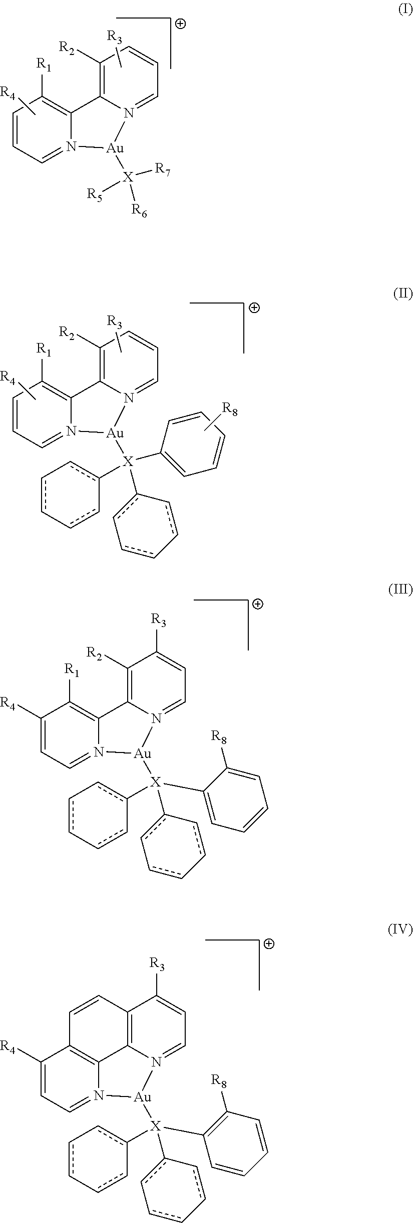

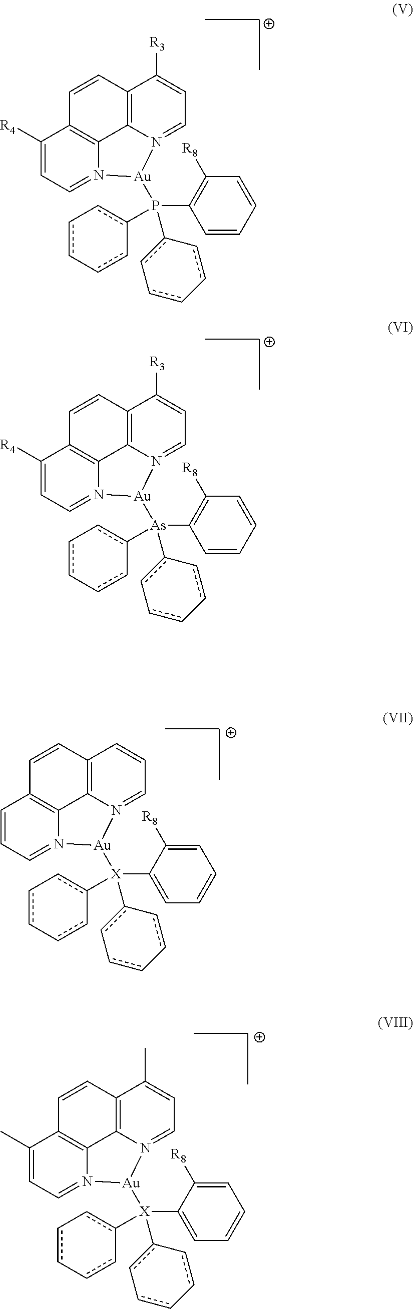

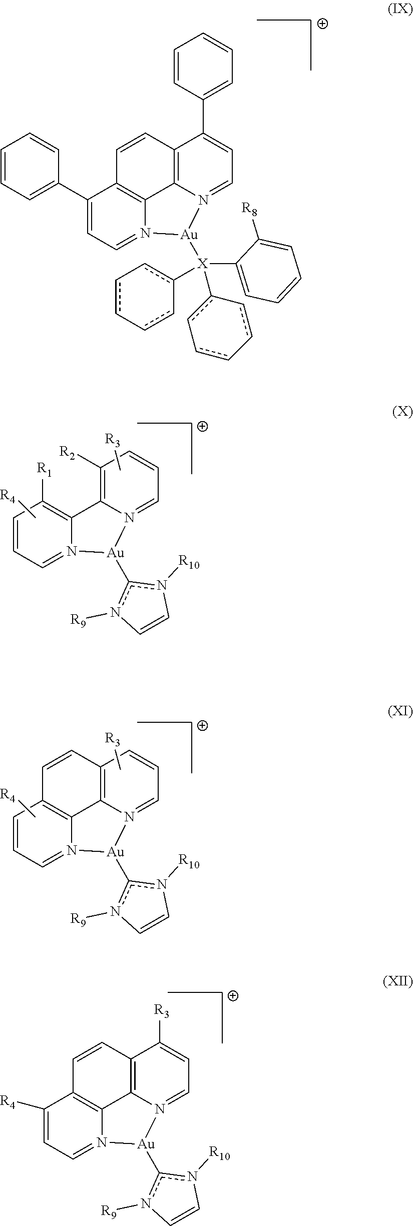

In formulae (I)-(XII),
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl;

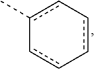

R₅ and R₆ are each or when X is C then R₅ and R₆ taken together with the C to which they are bound can form a 5-membered ring that is substituted or unsubstituted;
R₇ is H or

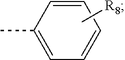

R₈ is selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

Some embodiments of the presently-disclosed subject matter include a pharmaceutical composition comprising a compound of any one of formulae (I)-(XII) and a pharmaceutically-acceptable carrier.

Some embodiments of the presently-disclosed subject matter include a method of conferring anti-cancer activity to a cancer cell, which involves contacting a cancer cell with an effective amount of a compound of any one of formulae (I)-(XII). In some embodiments, conferring anti-cancer activity results in one or more of: inhibiting proliferation of the cancer cell, inhibiting metastasis, and killing the cancer cell. In some embodiments of the method, the cell is a cultured cell. In some embodiments of the method, the cell is in a subject. In some embodiments of the method, the subject is a mammal.

Some embodiments of the presently-disclosed subject matter include the use of a compound of any one of formulae (I)-(XII) in a medicament for the treatment of a cancer.

Some embodiments of the presently-disclosed subject matter include a method of modulating mitochondrial function in a cell, which involves contacting a cell with an effective amount of a compound of any one of formulae (I)-(XII). In some embodiments of the method, the cell is a cancer cell. In some embodiments of the method, the cell is a cultured cell. In some embodiments of the method, the cell is in a subject. In some embodiments of the method, the subject is a mammal.

Some embodiments of the presently-disclosed subject matter include the use of a compound of any one of formulae (I)-(XII) in a medicament for the treatment of a condition involving mitochondrial dysfunction.

Some embodiments of the presently-disclosed subject matter include a method of increasing reactive oxygen species (ROS) in a cell, which involves contacting a cell with an effective amount of a compound of any one of formulae (I)-(XII). In some embodiments of the method, the effective amount is from about 10 nM to about 100 uM. In some embodiments of the method, the cell is a cancer cell. In some embodiments of the method, the cell is a cultured cell. In some embodiments of the method, the cell is in a subject. In some embodiments of the method, the subject is a mammal.

Some embodiments of the presently-disclosed subject matter include the use of a compound of any one of formulae (I)-(XII) in a medicament for increasing reactive oxygen species (ROS) in a cell.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 illustrates how mitochondrial biogenesis is a key component in maintaining cell homeostasis.

FIGS. 2A-2G includes X-ray crystal structures of AuTri-3 (FIG. 2A), AuTri-4 (FIG. 2B), AuTri-6 (FIG. 2C), AuTri-8 (FIG. 2D), AuTri-9 (FIG. 2E), AuTri-10 (FIG. 2F), and Tri-C (FIG. 2G). Outer-sphere solvent molecules are omitted for clarity. Thermal ellipsoids are shown at the 50% probability level with disorder at the gold center.

FIGS. 3A-3P include the results of cell proliferation assays. Cell viability of all 10 complexes in MRCS over 72 h (FIG. 3A). Cell viability of Sphos derivatives in MDA-MB-231 over 72 h (FIG. 3B). Cell viability of AuTri-9 in a panel of cell lines over 72 h (FIG. 3C). SAR of Sphos — based derivatives in MDA-MB-231 over 72 h (FIG. 3D). Cell survival of AuTri-1 in MDA-MB-231 (FIG. 3E). Cell survival of AuTri-2 in MDA-MB-231 (FIG. 3F). Cell survival of AuTri-3 in MDA-MB-231 (FIG. 3G). Cell survival of AuTri-4 in MDA-MB-231 (FIG. 3H). Cell survival of AuTri-5 in MDA-MB-231 (FIG. 3I). Cell survival of AuTri-6 in MDA-MB-231 (FIG. 3J). Cell survival of AuTri-7 in MDA-MB-231 (FIG. 3K). Cell survival of AuTri-8 in MDA-MB-231 (FIG. 3L). Cell survival of AuTri-9 in MDA-MB-231 (FIG. 3M). Cell survival of AuTri-10 in MDA-MB-231 (FIG. 3N). Cell survival of Tri-C in MDA-MB-468 and HCC-1937 (FIG. 3O). Cell survival of bidentate ligands, Auranofin, Cisplatin, and NaSbF6 as controls in MDA-MB-231 (FIG. 3P). All data are plotted as the mean ±s.e.m., n=3.

FIGS. 4A-4E include UV-Vis assessment of AuTri-1-10 stability and reactivity. UV-Vis stability of AuTri-9 (50 μM) in DMEM at 37 ° C. over 24 hours (FIG. 4A). UV-Vis stability of AuTri-9 (50 μM) in DMSO at 37° C. over 24 hours (FIG. 4B). UV-Vis stability of AuTri-9 (50 μM) in PBS at 37° C. over 24 hours (FIG. 4C). Reactivity of AuTri-9 (50 μM) with NAC at 37° C. over 1 h (FIG. 4D). Reactivity of AuTri-9 (50 μM) with GSH at 37° C. over 1 h (FIG. 4E).

FIG. 5A includes images from transmission electron microscopy of known cell death inducers, vehicle control, and AuTri-9 in MDA-MB-231, 1% DMSO was added to the control wells. Depicted in the panels as follows: healthy mitochondria (white arrows with vehicle, staurosporine, and H₂O₂), fragmented cristae (gray arrows with AuTri-9), cytosolic swelling (white star with H₂O₂), shrunken mitochondria (arrow and box with erastin), double membrane vesicle (gray arrows with rapamycin.

FIG. 5B presents maximal cristae width, where data are representative of 10 cells chosen at random n=10, where mitochondria were also chosen at random, and a maximum of 3 cristae measured per individual mitochondrion. Total number of cristae measured per each cell, n=100, data are then plotted as mean ±s.e.m.,

FIG. 5C includes immunoblots of OPA1, MFF, MFN1, and TOM20.

FIGS. 5D-5F present representative quantitative protein content of OPA1, MFN1, and TOM20, respectively.

FIG. 6 illustrates intracellular ROS accumulation in MDA-MB-231 monitored by DCF-DA fluorescence using FACS. AuTri-9 was used at a concentration of 5 μM for the designated time points. TBHP was used as a positive control. 1% DMSO was used as the vehicle control. Data are plotted as the mean ±s.d., n=3, n.s.=not significant, * p <0.01.

FIG. 7 includes results from quantitative proteomics in MDA-MB-231 upon treatment with AuTri-9.

FIG. 8A includes data from a mitostress test performed in MDA-MB-231 with AuTri-9 (injected pneumatically), data are plotted as mean ±s.d. n=8.

FIG. 8A provides metabolic parameters derived from the Mitostress test, data are plotted as mean ±s.d. n=8.

FIG. 8C presents JC-1 fluorimetric determination of MMP in MDA-MB-231 using AuTri-9, images are representative of 3 separate replicates.

FIG. 8D includes immunoblotting of SOD1, SOD2, NRF2, and Cytochrome C.

FIGS. 8E-8H present representative quantitative protein content of SOD1, SOD2, NRF2 and Cytochrome C respectively, n=5.

FIG. 9A-9B present cellular uptake of AuTri-9 using GF-AAS in MDA-MB-231. Subcellular uptake of AuTri-9 in MDA-MB-231 at 1 μM for 6 hours, data are plotted as the mean ±s.d., n=3 (FIG. 9A). Uptake inhibition using known inhibitors 1 hour prior to treatment with AuTri-9 (1 μM, 6 hours), data are plotted as the mean ±s.d., n=3 (FIG. 9B).

FIG. 10 presents in vivo toxicity as determined by mouse body weight. Female athymic Nu/Nude mice were treated with AuTri-9 or vehicle control at 10 mg/kg twice a week via intraperitoneal injection. n=2.

FIGS. 11A-11C include results of a in vivo study to determine the effects of Tri-C on glioblastoma xenograft tumor volume. BALB/c Nude mice were treated with Tri-C, Cisplatin, or vehicle control twice a week for 28 days via intraperitoneal injection. Normalized body weight as a function of time (FIG. 11A) and tumor volume as a function of time (FIG. 11B) are presented. Images of mice treated with Tri-C, Cisplatin, or vehicle control at Day 0, 9, 19, and 25 are provided (FIG. 11C).

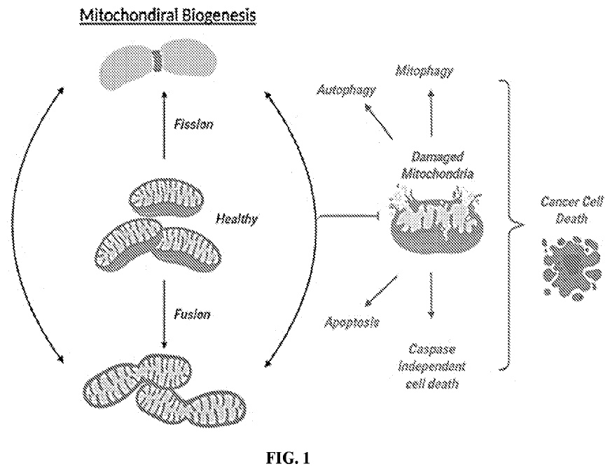

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes a compound that is a tri-coordinate Au(I) complexes, or a pharmaceutically acceptable salt thereof. The presently-disclosed subject matter also includes a pharmaceutical composition including tri-coordinate Au(I) complex as disclosed herein and a pharmaceutically-acceptable carrier. The presently-disclosed subject matter also includes methods of conferring anti-cancer activity to a cancer cell. The presently-disclosed subject matter also includes use of a compound disclosed herein in a medicament for the treatment of a cancer. The presently-disclosed subject matter also includes methods of modulating mitochondrial function in a cell. The presently-disclosed subject matter also includes use of a compound disclosed herein in a medicament for the treatment of a condition involving mitochondrial dysfunction. The presently-disclosed subject matter also includes methods of increasing reactive oxygen species (ROS) in a cell. The presently-disclosed subject matter also includes use of a compound disclosed herein in a medicament for increasing reactive oxygen species (ROS) in a cell.

Some embodiments of the presently-disclosed subject matter include a compound of the following formula (I), or a pharmaceutically-acceptable salt thereof

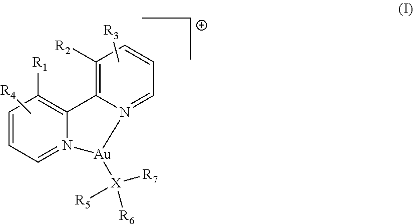

In formula (I),
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl;
R₅ and R₆ are each

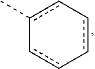

or when X is C then R₅ and R₆ taken together with the C to which they are bound can form a 5-membered ring that is substituted or unsubstituted;
R₇ is H

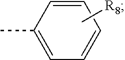

and
R₈ is selected from the group consisting of H, alkyl, cycloalkyl, aryl, and heterocycloalkyl.

Some embodiments of the presently-disclosed subject matter include a compound of the following formula (II), or a pharmaceutically-acceptable salt thereof

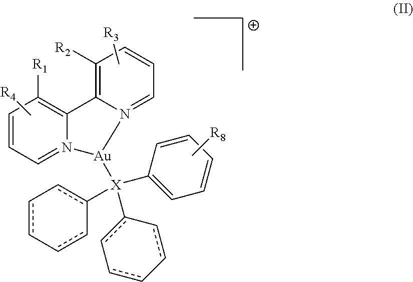

In formula (II),
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

Some embodiments of the presently-disclosed subject matter include a compound of the following formula (III), or a pharmaceutically-acceptable salt thereof

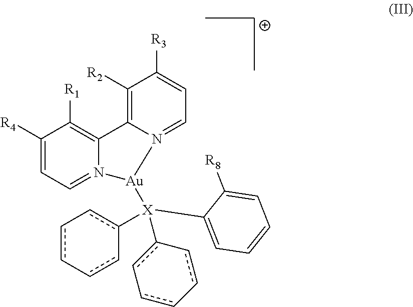

In formula (III),
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

Some embodiments of the presently-disclosed subject matter include a compound of the following formula (IV), or a pharmaceutically-acceptable salt thereof.

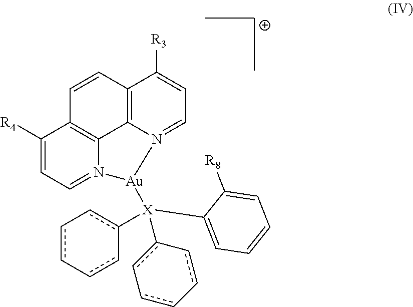

In formula (IV),
X is C, P or As;
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

Some embodiments of the presently-disclosed subject matter include a compound of the following formula (V), or a pharmaceutically-acceptable salt thereof

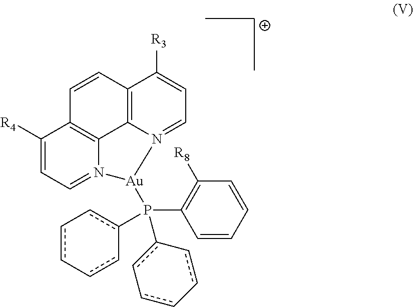

In formula (V),
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

Some embodiments of the presently-disclosed subject matter include a compound of the following formula (VI), or a pharmaceutically-acceptable salt thereof

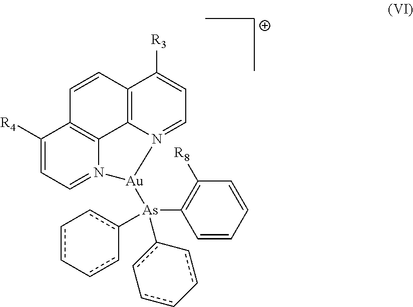

In formula (VI),
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

Some embodiments of the presently-disclosed subject matter include a compound of the following formula (VII), or a pharmaceutically-acceptable salt thereof

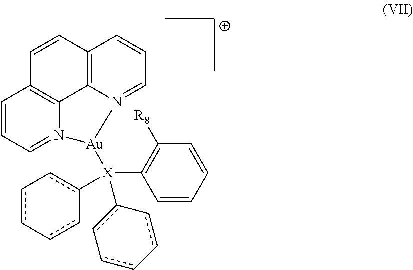

In formula (VII), R₈ is selected from the group consisting of H, alkyl, cycloalkyl, aryl, and heterocycloalkyl.

Some embodiments of the presently-disclosed subject matter include a compound of the following formula (VIII), or a pharmaceutically-acceptable salt thereof

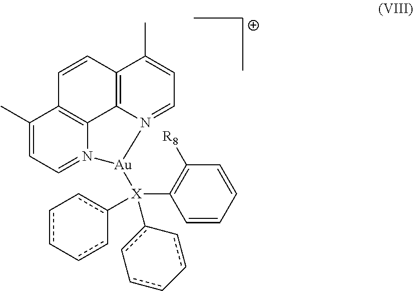

In formula (VIII), R₈ is selected from the group consisting of H, alkyl, cycloalkyl, aryl, and heterocycloalkyl.

Some embodiments of the presently-disclosed subject matter include a compound of the following formula (IX), or a pharmaceutically-acceptable salt thereof

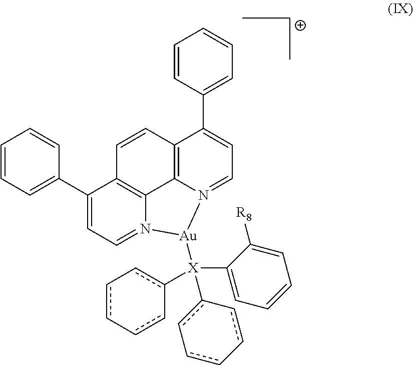

In formula (IX), R₈ is selected from the group consisting of H, alkyl, cycloalkyl, aryl, and heterocycloalkyl.

Some embodiments of the presently-disclosed subject matter include a compound of the following formula (X), or a pharmaceutically-acceptable salt thereof

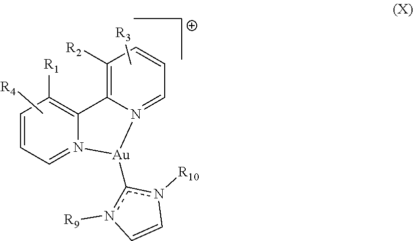

In formula (X),
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

Some embodiments of the presently-disclosed subject matter include a compound of the following formula (XI), or a pharmaceutically-acceptable salt thereof

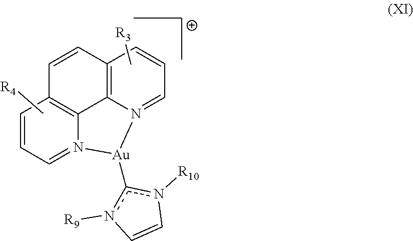

In formula (XI),
R₃ and R₄ are independently selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

Some embodiments of the presently-disclosed subject matter include a compound of the following formula (XII), or a pharmaceutically-acceptable salt thereof

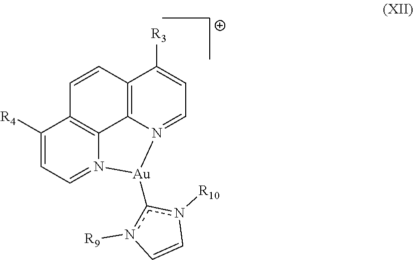

In formula (XII),
R₃ and R₄ are independently selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

Some embodiments of the presently-disclosed subject matter include a compound, or a pharmaceutically-acceptable salt thereof, of one of the following formulae:

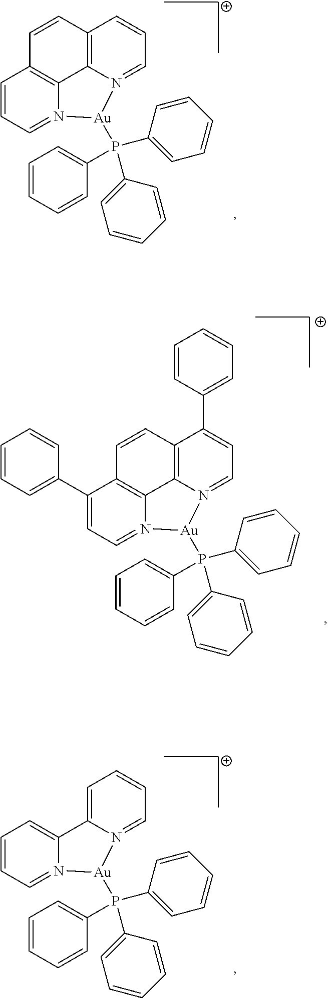

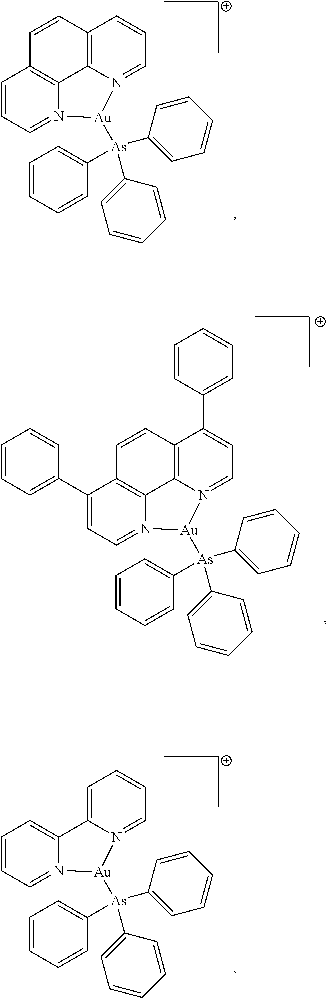

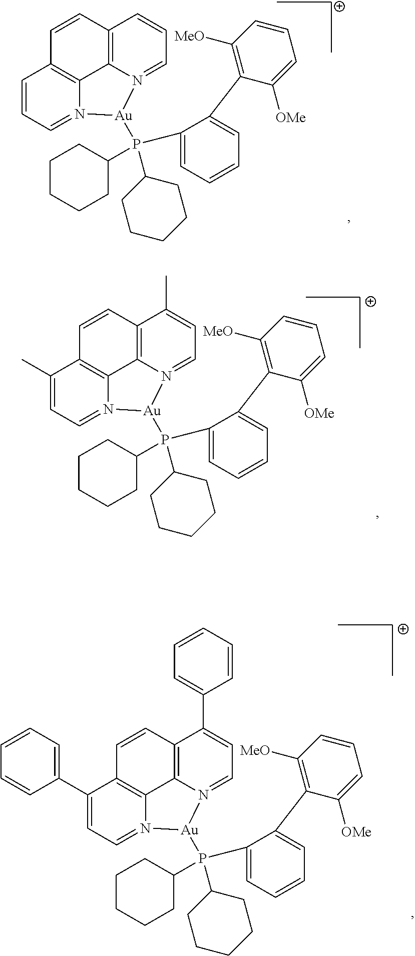

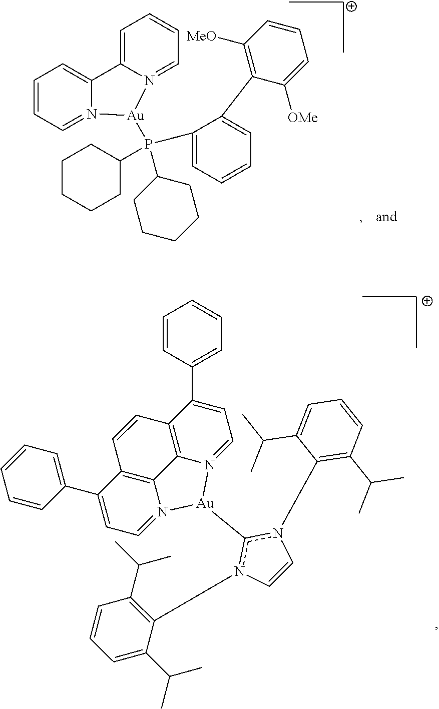

Some embodiments of the presently-disclosed subject matter include a pharmaceutical composition comprising a compound of any one of formulae (I)-(XII) and a pharmaceutically-acceptable carrier.

Some embodiments of the presently-disclosed subject matter include a method of conferring anti-cancer activity to a cancer cell, which involves contacting a cancer cell with an effective amount of a compound of any one of formulae (I)-(XII). In some embodiments, conferring anti-cancer activity results in one or more of: inhibiting proliferation of the cancer cell, inhibiting metastasis, and killing the cancer cell. In some embodiments of the method, the cell is a cultured cell. In some embodiments of the method, the cell is in a subject. In some embodiments of the method, the subject is a mammal.

Some embodiments of the presently-disclosed subject matter include the use of a compound of any one of formulae (I)-(XII) in a medicament for the treatment of a cancer.

Some embodiments of the presently-disclosed subject matter include a method of modulating mitochondrial function in a cell, which involves contacting a cell with an effective amount of a compound of any one of formulae (I)-(XII). In some embodiments of the method, the cell is a cancer cell. In some embodiments of the method, the cell is a cultured cell. In some embodiments of the method, the cell is in a subject. In some embodiments of the method, the subject is a mammal.

Some embodiments of the presently-disclosed subject matter include the use of a compound of any one of formulae (I)-(XII) in a medicament for the treatment of a condition involving mitochondrial dysfunction.

Some embodiments of the presently-disclosed subject matter include a method of increasing reactive oxygen species (ROS) in a cell, which involves contacting a cell with an effective amount of a compound of any one of formulae (I)-(XII). In some embodiments of the method, the effective amount is from about 10 nM to about 100 μM. In some embodiments of the method, the cell is a cancer cell. In some embodiments of the method, the cell is a cultured cell. In some embodiments of the method, the cell is in a subject. In some embodiments of the method, the subject is a mammal.

Some embodiments of the presently-disclosed subject matter include the use of a compound of any one of formulae (I)-(XII) in a medicament for increasing reactive oxygen species (ROS) in a cell.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

As used in the specification and the appended claims, the singular forms “a,” “an” and “the” include plural referents unless the context clearly dictates otherwise. Thus, for example, reference to “a cell,” “a functional group,” “an alkyl,” or “a residue” includes mixtures of two or more such cells, functional groups, alkyls, or residues, and the like.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “subject” refers to a target of administration or medical procedure. The subject of the herein disclosed methods can be a human or animal. The subject may also be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can favorably inhibit or kill cancer cells.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO — moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as -OA¹ where A¹ is alkyl or cycloalkyl as defined above. “Alkoxy” also includes polymers of alkoxy groups as just described; that is, an alkoxy can be a polyether such as -OA¹-A² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by a formula —C(O)H. Throughout this specification “C(O)” is a short hand notation for a carbonyl group, i.e., C═O.

The terms “amine” or “amino” as used herein are represented by a formula NA¹A² A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “carboxylic acid” as used herein is represented by a formula —C(O)OH.

The term “ester” as used herein is represented by a formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by a formula —(A¹O(O)C-A²-C(O)O)ₐ— or —(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by a formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by a formula —(A¹OA²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole,thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by a formula —OH.

The term “ketone” as used herein is represented by a formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by a formula —N₃.

The term “nitro” as used herein is represented by a formula —NO₂. The term “nitrile” as used herein is represented by a formula —CN.

The term “silyl” as used herein is represented by a formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by a formulas —S(O)A¹, —S(O)₂A¹, —OS(O)2A¹, or —OS(O)₂A¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by a formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by a formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by a formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by a formula —SH.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compounds disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

In some aspects, a structure of a compound can be represented by a formula:

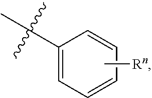

which is understood to be equivalent to a formula:

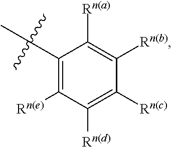

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance. Likewise, when a group R is defined as four substituents, R is understood to represent four independent substituents, R^{a}, R^{b}, R^{c}, and R^{d}. Unless indicated to the contrary, the substituents are not limited to any particular order or arrangement.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspect or combination of aspects of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

The presently-disclosed subject matter is further illustrated by the following specific but non-limiting examples. The following examples may include compilations of data that are representative of data gathered at various times during the course of development and experimentation related to the present invention.

### EXAMPLES

Mitochondrial structure and organization is integral to maintaining mitochondrial homeostasis and an emerging biological target in ageing, inflammation, neurodegeneration, and cancer. The study of mitochondrial structure and its functional implications remains challenging in part due to lack of available tools for direct engagement, particularly in a disease setting. In the studies described in these examples, a gold-based approach is disclosed to perturb mitochondrial structure in cancer cells.

The design and synthesis of a series of tri-coordinate Au(I) complexes with systematic modifications to group 15 non-metallic ligands establish structure-activity relationships to identify physiologically relevant tools for mitochondrial perturbation. An exemplary compound, AuTri-9, is shown to selectively disrupts breast cancer mitochondrial structure rapidly as observed by transmission electron microscopy with attendant effects on fusion and fission proteins. This phenomenon triggers severe depolarization of the mitochondrial membrane in cancer cells. The high in vivo tolerability of the exemplary compound in mice demonstrates its utility. This work provides a basis for rational design of gold-based agents to control mitochondrial structure and dynamics.

### Example 1: Compounds and Synthesis

AuTri Complexes. The quest to develop chemical tools that target distinct aspects of the mitochondrion prompted investigations into reagents that can perturb mitochondrial structure. Given previous reports in the literature of cationic gold complexes directly affecting mitochondrial damage and mitochondrial permealization,³³⁻⁴⁰ exploring the effect of cationic tri-coordinate complexes in mitochondrial biological systems was of interest.

Tri-coordinate compounds can assume an unsymmetrical structure with one longer metal-heteroatom bond length, which is weak enough to act as a coordination site upon bond breakage. This deviation from the use of leaving groups bonded to metal centers considered in connection with these studies. It was contemplated that unsymmetrical gold compounds would provide a distinct labile character to interact with biomolecules (vide infra). Therefore, a class of unique compounds was designed and synthesized to enable SAR studies. Tri-coordinate gold(I) complexes were achieved by a facile synthetic protocol (Scheme 1).

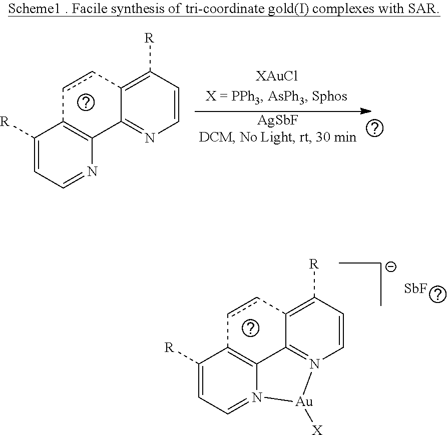

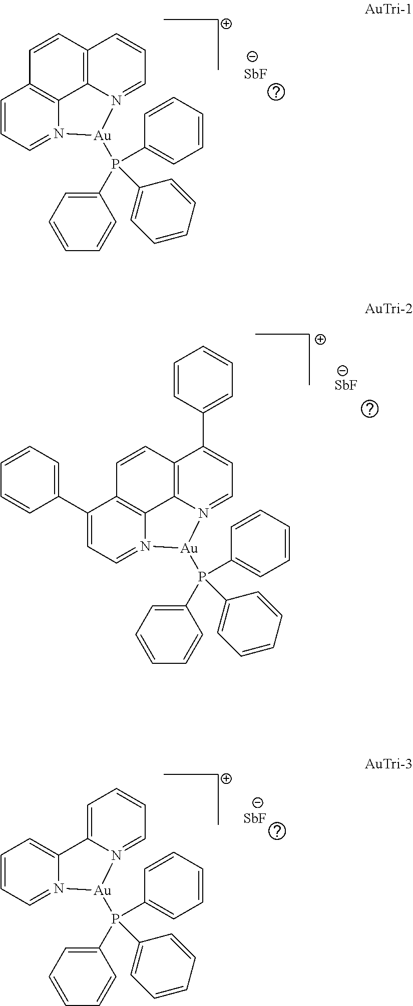

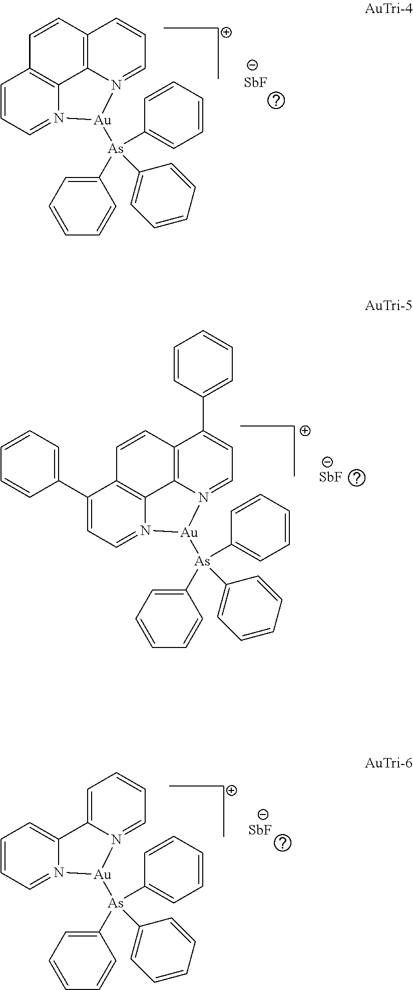

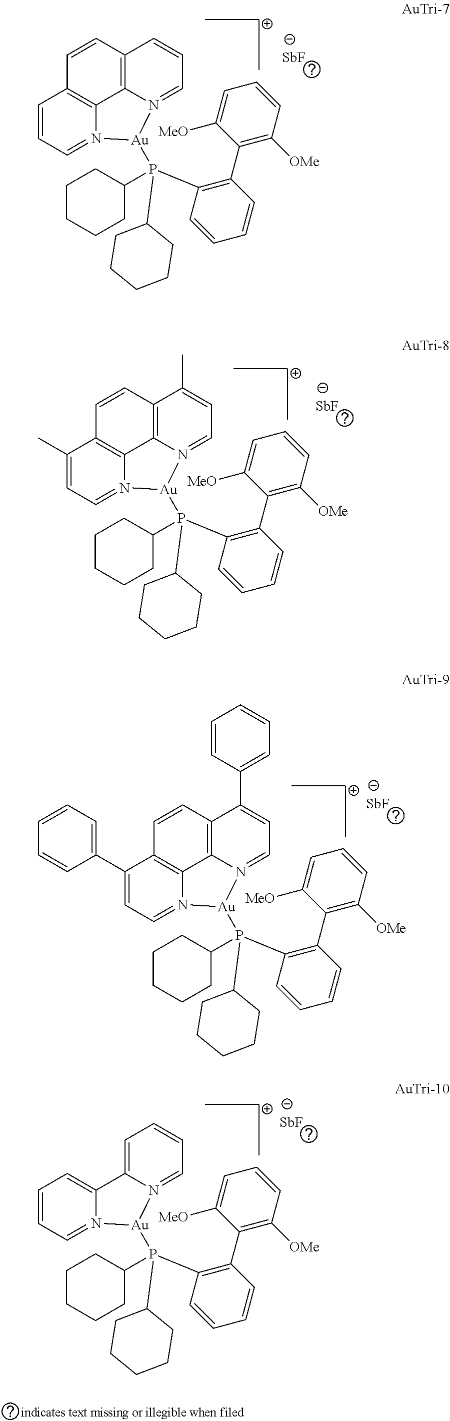

Commercially available phosphine and arsine ligands were treated with either HAuCl₄·3H₂O or AuCl(tht) to afford the neutral gold(I) precursors. These precursors were subjected to silver transmetalation by adding to a suspension of the gold(I) pecursor in dichloromethane, the corresponding silver salt, and NAN bidentate ligand of choice to afford a library of exemplary cationic tri-coordinate gold(I) complexes AuTri-1-10 in good-to-excellent yields (data not shown). The synthetic feasibility allows for a vast library of complexes to be developed in a short amount of time.

General Experimental Details: All reactions were carried under ambient conditions in air unless otherwise noted. Solvents were of ACS grade (Pharmco-Aaper) and used as is. The starting Au(I) complexes (ClAu(tht), ClAuPPh3, ClAuAsPh3, and ClAuSphos) were prepared from procedures in the literature.⁶⁹⁻⁷² Triphenylphosphine, triphenylarsine, and tetrahydrothiophene were purchased from Alfa Aesar. Sphos and AgSbF6 were purchased from Strem Chemicals and stored under N2. Phenanthroline (phen), 4,7-dimethylphenanthroline (dmphen), bethophenanthroline (bphen), and bipyridine (bpy) were all purchased from Matrix Scientific. Deuterated solvents were purchased from Cambridge Isotope Laboratories (Andover, MA). 3-(4,5-dimethylthiazol-2-yl)-2,5-diphenyltetrazolium bromide (MTT) and JC-1 were purchased from Cayman Chemicals. NMR spectra were recorded on a Bruker Avance NEO 400 MHz spectrometer and samples calibrated for: ¹H NMR (CD3CN δ=1.94 ppm and DMSO-d6 δ=2.50 ppm), ¹³C NMR (CD3CN 6=118.26 and 1.32 ppm and DMSO-d6 6=49.00 ppm), and ¹H NMR externally referenced to _{H3PO4}δ=0.00). High-resolution mass spectra (HRMS) were obtained by direct flow injection (injection volume=2 μL) using ElectroSpray Ionization (ESI) on a Waters Synapt G2 HDMS instrument in the positive mode with a quadripole/TOF analyzer (UC Boulder). In addition to spectroscopic characterization, the purity of all compounds was assessed by RP-HPLC using an Agilent Technologies 1100 series HPLC instrument and an Agilent Phase Eclipse Plus C18 column (4.6 mm' 100 mm; 3.5 μm particle size). All compounds were found to be 397% pure.

### General Procedure for the Preparation of Au(I) Tricoordinate

Complexes. To 5 mL of DCM was added 1 equivalent of the corresponding NAN bidentate ligand and 1.1 equivalents of AgSbF6 in a 20 mL screw cap vial wrapped in aluminum foil. This mixture was stirred for 5 minutes at room temperature where it was then added to a solution of 1 equivalent of corresponding XAu(I)Cl (X=PPh3, AsPh3, and Sphos). The mixture was then stirred for 30 minutes at room temperature under the exclusion of light. The solution was then placed in 1.5 mL Eppendorf tubes and centrifuged at high RPM to pellet the AgCl(S). The supernatant was decanted into a round-bottom flask (cleaned with aqua regia) and concentrated in vacuo at 40° C. Excess Et2O was added to precipitate the corresponding tri-coordinate complex, which was further washed with excess Et2O and vacuum dried under the exclusion of light. All complexes except AuTri-4-6 are benchtop stable indefinitely. AuTri-4-6 will slowly degrade into a purple looking solid at room temperature, indicating reduction of the gold metal center. In solution phase, (DMSO and MeCN), AuTri-4-6 will rapidly decompose and deposit elemental gold on the glass vial in both air and under nitrogen atmosphere. Storage at +4° C. significantly improves the longevity of the complexes AuTri-1-3 and AuTri-7-10.

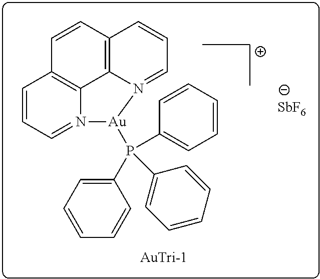

AuTri-1: 1H NMR (400 MHz, MeCN-d3) 9.25 (d, J=4 Hz, 2H), 8.92 (d, J=8 Hz, 2H), 8.30 (s, 2H), 8.20 (q, J=8 Hz, 2H) 7.86-7.80 (m, 6H), 7.72-7.65 (m, 9H); 13C NMR (101 MHz, DMSO-d6) δ=152.35, 142.29, 140.43, 134.42, 134.28, 132.74, 132.71, 130.20, 130.08, 129.40, 128.06, 126.18; ³¹P NMR (162 MHz, DMSO-d6) δ=31.27. Purity was demonstrated to be >97% by RP-HPLC: Rf=5.09 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

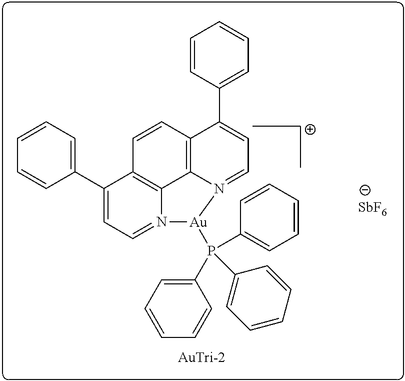

AuTri-2: 1H NMR (400 MHz, DMSO-d6) 8.13 (d, J=8 Hz, 2H), 8.05 (s, 2H), 7.79-7.66 (m, 25H); 13C NMR (101 MHz, DMSO-d6) 6=152.00, 151.53, 143.04, 136.30, 134.40, 132.73, 132.71, 130.21, 130.09, 129.58, 127.50, 126.35, 125.53; ³¹P NMR (162 MHz, DMSO-d6) δ=39.12. Purity was demonstrated to be >97% by RP-HPLC: Rf=10.15 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

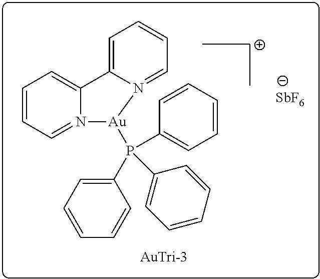

AuTri-3: ¹H NMR (400 MHz, MeCN-d3) 8.81 (bs, 2H), 8.51 (d, J=8 Hz, 2H), 8.27 (bs, 2H), 7.81 (bs, 2H) 7.60-7.51 (m, 15H); ¹³C NMR (101 MHz, MeCN-d3) 6=142.29, 134.83, 133.84,132.64, 132.46, 132.39, 131.61, 131.15, 130.77, 130.54, 127.50, 124.40, 124.10; ³¹P NMR (162 MHz, DMSO-d6) δ=30.75. Purity was demonstrated to be >97% by RP-HPLC: Rf=4.77 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

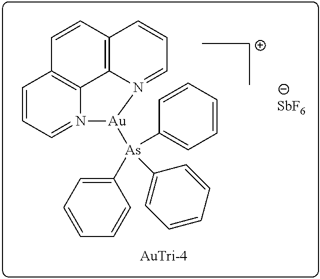

AuTri-4: ¹H NMR (400 MHz, DMSO-d₆) 8.82 (d, J=8 Hz, 2H), 8.59 (d, J=8 Hz, 2H), 8.23 (t, J=8 Hz, 2H), 7.77 (t, J=8 Hz, 2H) 7.68-7.62 (m, 15H).

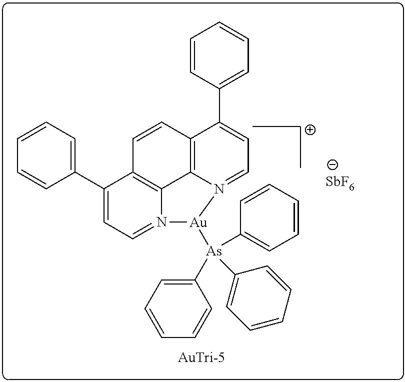

AuTri-5: ¹H NMR (400 MHz, DMSO-d6) 9.29 (d, J=4 Hz, 2H), 8.14 (d, J=4 Hz, 2H), 8.07 (s, 2H), 7.76 (t, J=4 Hz, 5H) 7.75-7.63 (m, 20H).

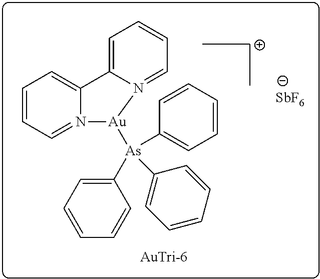

AuTri-6: ¹H NMR (400 MHz, DMSO-d6) 8.81 (d, J=8 Hz, 2H), 8.59 (d, J=

8 Hz, 2H), 8.23 (t, J=8 Hz, 2H), 7.77 (t, J=8 Hz, 2H) 7.68-7.62 (m, 15H).

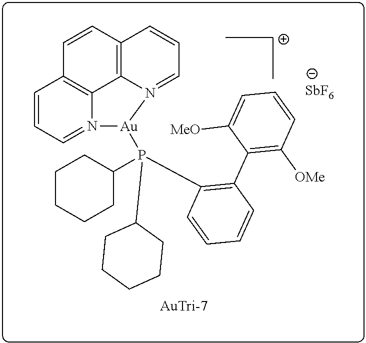

AuTri-7: ¹H NMR (400 MHz, DMSO-d6) 8.91 (d, J=8 Hz, 4H), 8.30 (s, 2H), 8.20 (t, J=8 Hz, 2H), 7.95 (t, J=8 Hz, 1H), 7.55 (quint., J=16 Hz, 2H), 6.96 (t, J=4 Hz, 2H), 5.72-5.61 (m, 3H), 3.38 (s, 6H), 2.14 (bs, 2H), 1.85 (bs, 4H), 1.73 (bs, 4H), 1.46-1.34 (m, 10H); ¹³C NMR (101 MHz, DMSO-d6) δ=156.13, 151.89, 151.54, 143.36, 143.23, 140.01, 139.04, 132.85, 132.34, 131.03, 127.82, 127.75, 125.76, 122.55, 105.64, 70.23, 32.02, 29.99, 26.24, 22.86, 21.87; 31 P NMR (162 MHz, DMSO-d6) δ=39.09. Purity was demonstrated to be >97% by RP-HPLC: Rf=5.22 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

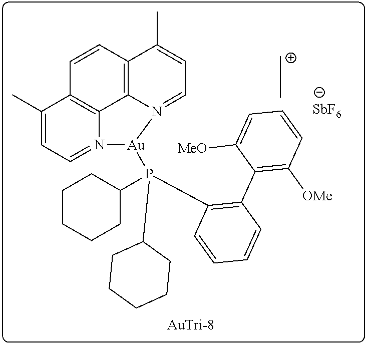

AuTri-8: 1 1-1NMR (400 MHz, DMSO-d6) 8.71 (d, J=8 Hz, 4H), 8.39 (s, 2H), 8.02 (t, J=8 Hz, 2H), 7.94 (t, J=8 Hz, 1H), 7.55 (quint., J=16 Hz, 2H), 6.96 (t, J=4 Hz, 1H), 5.76-5.61 (m, 3H), 3.39 (s, 6H), 2.95 (s, 6H), 2.16 (bs, 2H), 1.84 (bs, 4H), 1.72 (bs, 4H), 1.48-1.27 (m, 10H); 13C NMR (101 MHz, DMSO-d6) δ=157.88, 151.11, 149.87, 142.87, 133.61, 133.53, 133.01, 132.97, 131.46, 129.75, 128.78, 128.16, 128.08, 126.23, 123.90, 103.32, 55.46, 32.03, 30.35, 26.80, 19.14; ³¹P NMR (162 MHz, DMSO-d6) δ=39.13. Purity was demonstrated to be >97% by RP-HPLC: Rf=6.01 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

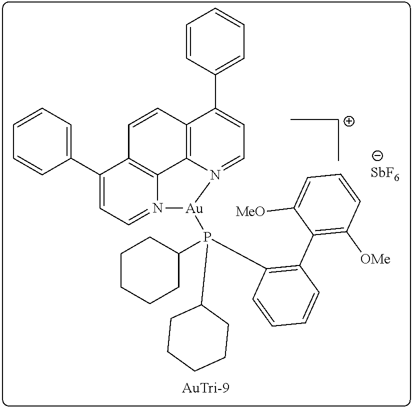

AuTri-9: ¹H NMR (400 MHz, DMSO-d6) 8.93 (bs, 2H), 8.18-8.12 (m, 6H), 7.98 (t, J=8 Hz, 2H), 7.94 (t, J=8 Hz, 1H), 7.70 (t, J=4 Hz, 11 H), 7.58 (quint., J=16 Hz, 1H), 7.00 (t, J=4 Hz, 1H), 5.86-5.67 (m, 3H), 3.44 (s, 6H), 2.20 (bs, 2H), 1.86-1.76 (m, 8H), 1.49-1.37 (m,10H); ¹³C NMR (101 MHz, DMSO-d6) δ=156.12, 152.47, 150.81, 143.28, 143.15, 140.39, 132.86, 132.78, 132.35, 132.31, 131.19, 128.80, 128.65, 127.83, 127.75, 126.46, 124.23, 122.61,122.54, 106.15,70.19, 31.88, 31.79, 29.80, 26.12, 22.72, 21.90; ³¹P NMR (162 MHz, DMSO-d6) δ=40.74. HRMS (m/z) calcd. 939.3354, found 939.3346 [M−SbF6]⁺, Purity was demonstrated to be >97% by RP-HPLC: Rf=10.87 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

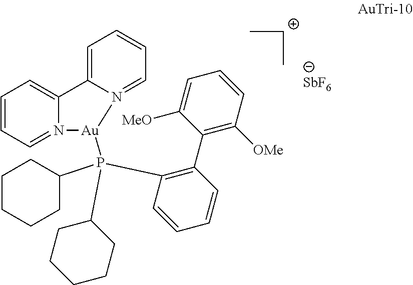

AuTri-10: ¹H NMR (400 MHz, DMSO-d6) 8.58 (bs, 2H), 8.49 (d, J=8 Hz, 2H), 8.24 (bs, 2H), 7.91 (quint., J=16 Hz, 1H), 7.80 (bs, 2H), 7.54 (d, J=8 Hz, 2H), 6.99 (t, J=8 Hz, 1H), 6.46 (bs, 1H), 6.02 (d, J=8 Hz, 2H), 3.43 (s, 6H), 2.03 (bs, 2H), 1.80-1.63 (m, 8H), 1.47-1.18 (m, 10H); ¹³C NMR (101 MHz, MeCN-d3) δ=157.97, 133.63, 133.56, 133.04, 133.01, 131.67, 130.31, 130.14, 129.62, 128.20, 128.13, 126.77, 126.04, 135.60, 103.80, 55.53, 31.69, 30.06, 26.87, 26.77, 26.63, 26.32; ³¹P NMR (162 MHz, DMSO-d6) δ=38.46. Purity was demonstrated to be >97% by RP-HPLC: Rf=4.82 minutes using the following method: Flow rate: 1 mL/min; λ=280 nm; Eluent A=H2O with 0.1% TFA; Eluent B=MeOH with 0.1% TFA; Solvent Gradient: 0-3 min (50:50 H2O: MeOH), 5 min (40:60 H2O: MeOH), 7 min (30:70 H2O: MeOH), 9 min (0:100 H2O: MeOH), 10 min (20:80 H2O: MeOH), 12 min until end of run (100:0 H2O: MeOH).

Tri-C: a synthetic protocol for Tri-C is provided in Scheme 2.

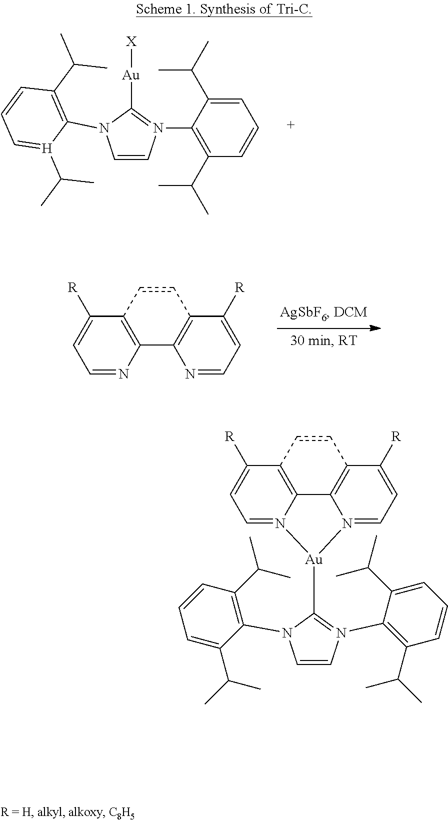

### Example 2: X-Ray Crystallography

To further elucidate the unique geometry, the structures of six complexes were solved by X-ray diffraction techniques. Crystals of all complexes were grown from slow diffusion of Et₂O into a concentrated solution of MeCN at room temperature. All crystals were mounted using polyisobutene oil on the end of a glass fibre, which had been mounted to a copper pin using an electrical solder. It was placed directly in the cold gas stream of a liquid nitrogen cryostat⁷³⁻⁷⁴ A Bruker D8 Venture diffractometer with graded multilayer focused MoKa X-rays (λ=0.71073 Å) was used to collect diffraction. Raw data were integrated, scaled, merged, and corrected for Lorentz-polarization effects using the APEX3 package. ⁷⁵⁻⁷⁷

Space group determination and structure solution and refinement were carried out with SHELXT and SHELXL respectively.⁷⁸⁻⁷⁹ All non-hydrogen atoms were refined with anisotropic displacement parameters. Hydrogen atoms were placed at calculated positions and refined using a riding model with their isotropic displacement parameters (Uiso) set to either 1.2Uiso or 1.5Uiso of the atom to which they were attached. Ellipsoid plots were drawn using SHELXTL-XP.⁸⁰ The structures, deposited in the Cambridge Structural Database, were checked for missed symmetry, twinning, and overall quality with PLATON,⁸¹ an R-tensor,⁸² and finally validated using CheckCIF.⁸¹

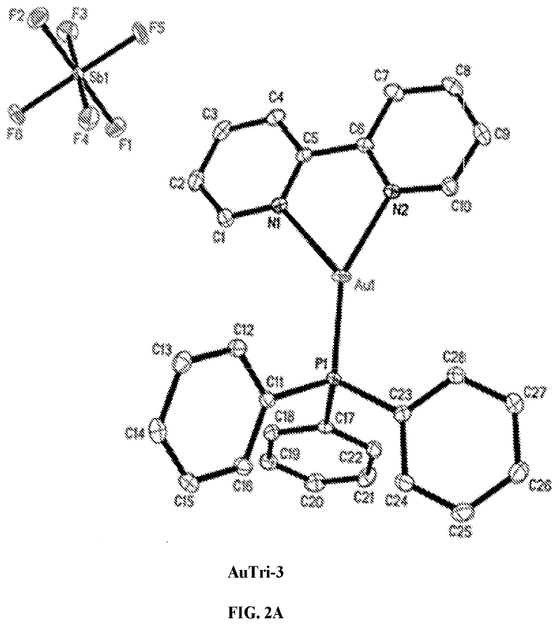

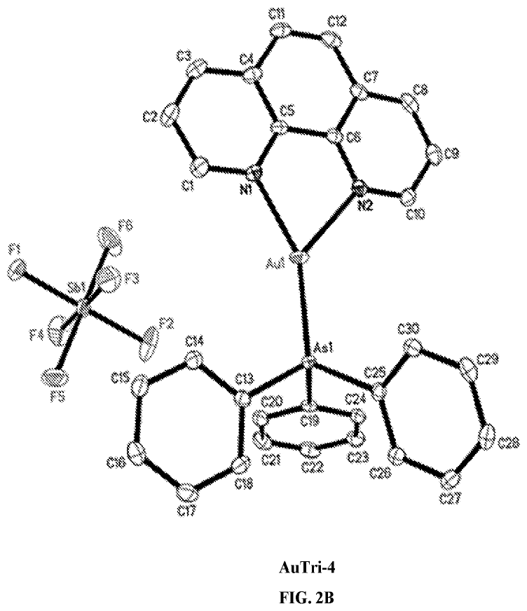

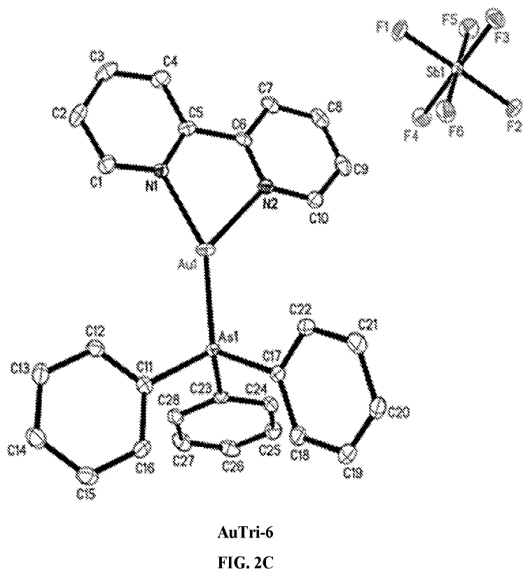

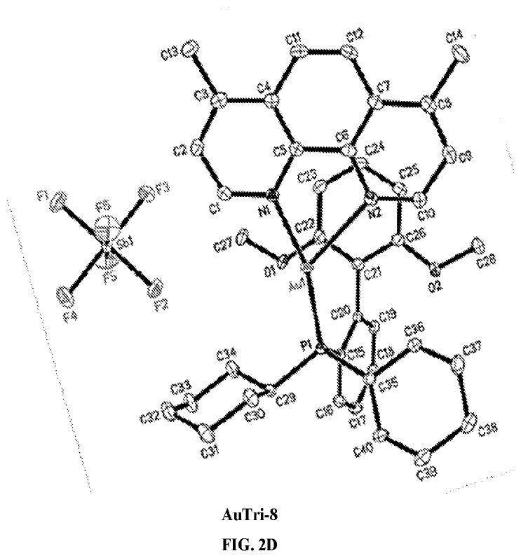

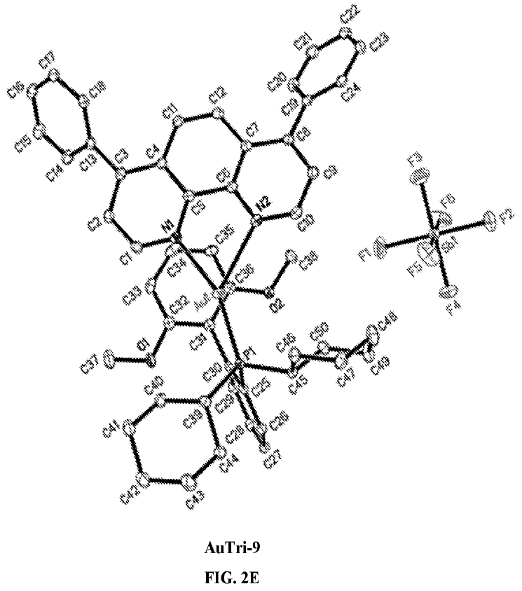

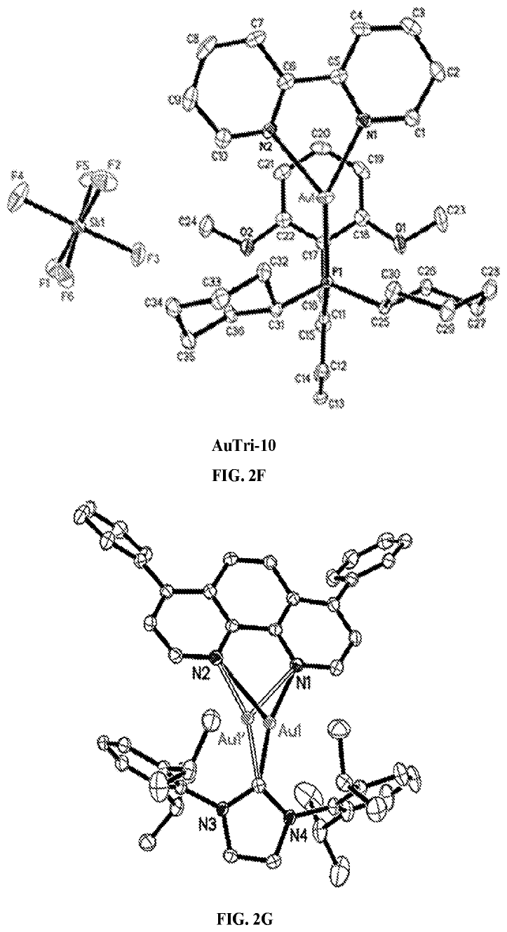

FIGS. 2A-2F include the structures of AuTri-3 (FIG. 2A), AuTri-4 (FIG. 2B), AuTri-6 (FIG. 2C), AuTri-8 (FIG. 2D), AuTri-9 (FIG. 2E), and AuTri-10 (FIG. 2F), and Tri-C (FIG. 2G).

The structure of AuTri-6 (FIG. 2C) demonstrates a clearly unsymmetrical three coordinate geometry around the gold center. The NAN ligand shows heavy distortion with one Au-N bond length significantly longer than the other as exemplified by compound AuTri-6 (Au1-N1-2.195(2) Å and Au1-N2-2.400(2) Å). Consequently, the bite angle between N1-Au-N2 (71.68° (7)) is significantly smaller than observed in gold(III) phenanthroline complexes, which are typically between 81 and 83°.⁴¹ Furthermore, as the coordination atom of the ancillary ligand moves down group 15, a slight increase of the distortion of the complex is observed. When AsPh3 is used in replacement, the Au1-N1 bond length is then 2.176(3) Å and Au-N2 is 2.403(2) Å.

The structure of AuTri-9 is provided in FIG. 2E. Again, the symmetric distortion between Au-N bonds can be observed with Au-N1 (2.1636(17) Å) and Au-N2 2.5046(17) Å). All further crystallographic parameters as well as structures for complexes AuTri-4, AuTri-8, and AuTri-10 can be found in FIGS. 2B, 2D, and 2F. The unique geometry of these complexes could impart unexplored reactivity in vivo in comparison to the typical linear dm gold complexes.

### Example 3: Cell Viability

Cell Culture. All cell lines were purchased from ATCC and routinely grown in a humidified incubator at 37° C. with 5-10% CO2. MDA-MB-468, MDA-MB-231, MDA-MB-175, MRCS, RPE-NEO, HCT116, and K562N were grown in DMEM supplemented with 10% FBS, 1% amphotericin and 1% penicillin/streptomycin. A2780 cells were grown in RPMI supplemented with 10% FBS, 1% amphotericin, and 1% penicillin/streptomycin, and 4 mM glutamine. All supplements along with PBS and trypsin-EDTA were purchased from Corning Inc. and used as is.

Cell Viability of AuTri-1-10 (Adherent Cell Lines). The cell viability of all 10 complexes were performed in MDA-MB-231. Additionally, AuTri-9 was evaluated in the following: MDA-MB-175, MA-MB-468, A2780, HCT116, OVCAR₈ and MRCS. Cells were grown to confluency and trypsin was added to detach and harvest cells. The cells were washed with 2 mL of PBS and suspended in 10 mL of the appropriate media. The cells were centrifuged at 2000 rpm for 5 minutes and the pellet washed with 2 mL of PBS then suspended in 5 mL of the appropriate media. The cells were plated at a density of 2,000 cells/well in a 96-well clear bottom plate and allowed to adhere overnight at 37° C. with 5-10% CO2. The compounds were prepared as a stock in DMSO and used fresh. The compounds were added at seven concentrations (<1% DMSO) with a 3× serial dilution starting at 50 μM for the highest concentration and incubated at 37° C. for 72 h with 5-10% CO2. 1% DMSO were added to control wells as the vehicle control. The medium was removed and a solution of MTT (100 μL, prepared by dissolving MTT at 5 mg/mL and diluting by 10x with DMEM) was added to each well and incubated for 4 h at 37° C. with 5-10% CO2. The dye was removed from each well and 100 μL of DMSO was added to induce cell lysis. The plates were read using a Genios plate reader (λ=570 nm).

Cell Viability of AuTri-9 (Suspended Cell Lines). The cell viability of AuTri-9 was determined in K562. Cells were grown to confluency and centrifuged at 2000 rpm for 5 minutes to collect the cell pellet. The cells were washed with 5 mL of PBS, suspended in 5 mL of DMEM, and centrifuged again at 2000 rpm for 5 minutes to collect the pellet. The pellet was then washed with 2 mL of PBS and suspended in 5 mL of DMEM. The cells were plated at density of 2,000 cells/well in a 96-well white bottom plate. AuTri-9 was prepared as a stock solution in DMSO and used fresh. The compound was added at seven concentrations (<1% DMSO) with a 3× serial dilution starting at 50 μM for the highest concentration and incubated at 37° C. for 72 h with 5-10% CO2. The cells were removed from the incubator and allowed to rest at room temperature for 30 minutes. To each designated well was then added 20 μL of CellTiter-Glo solution and orbitally shaken for 5 minutes and the luminescence (1000 ms integration and 150 ms gain) acquired on a Genios plate reader.

In Vitro Cell Viability. The anticancer efficacy of the library of the tri-coordinate gold(I) complexes was explored in vitro by initially testing the complexes in a cisplatin resistant breast cancer cell line, MDA-MB-231 (Table 1). Over 50% of cancer patients world-wide receive some type of platinum based drug. ⁴²

TABLE 1
Cell viability of all ten complexes in TNBC cell
line MDA-MB-231 using MTT assay. Cells were incubated
with the compounds for 72 hours. All compounds were
added from stock solutions at a DMSO concentration <1%.
Data are represented as the mean ± s.e.m., n = 3
MDA - MB - 231

| | IC₅₀ |
|---|---|
| Compound | Value (μM) |
| AuTri-1 | 8.65 ± 0.122 |
| AuTri-2 | 2.51 ± 0.151 |
| AuTri-3 | 6.56 ± 0.354 |
| AuTri-4 | 3.98 ± 0.359 |
| AuTri-5 | 0.825 ± 0.065 |
| AuTri-6 | 11.75 ± 0.492 |
| AuTri-7 | 5.41 ± 0.245 |
| AuTri-8 | 1.12 ± 0.095 |
| AuTri-9 | 0.501 ± 0.03 |
| AuTri-10 | 4.65 ± 0.423 |
| 1,10-phenantrholine | 7.21 ± 1.68 |
| 4,7-dimethylphenanthroline | 14.08 ± 2.58 |
| Bathophenanthroline | 7.48 ± 1.64 |
| Bipyridine | 17.54 ± 0.354 |
| Cisplatin | 34.56 ± 0.783 |
| Auranofin | 1.31 ± 0.15 |
| NaSbF₆ | >50 |

These drugs have been used to treat testicular, ovarian, cervical, and non-small-cell lung cancers effectively. However, the success of these drugs are limited by resistance from long exposure times to the drugs.⁴³ Moreover, platinum based drugs have been used in breast cancer cell lines such as BRCA-1 deficient and triple negative cell lines; however, their efficacy is short lived due to resistance from alteration to multiple cellular pathways.⁴⁴⁻⁴⁵ With this in mind, the initial screening of the tri-coordinate complexes in MDA-MB-231 proved to be a useful first step in evaluating the cellular toxicity.

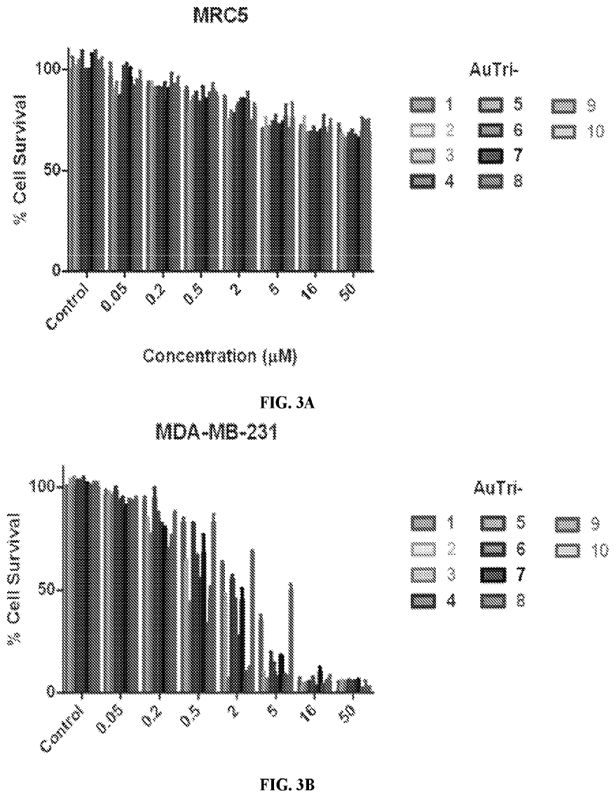

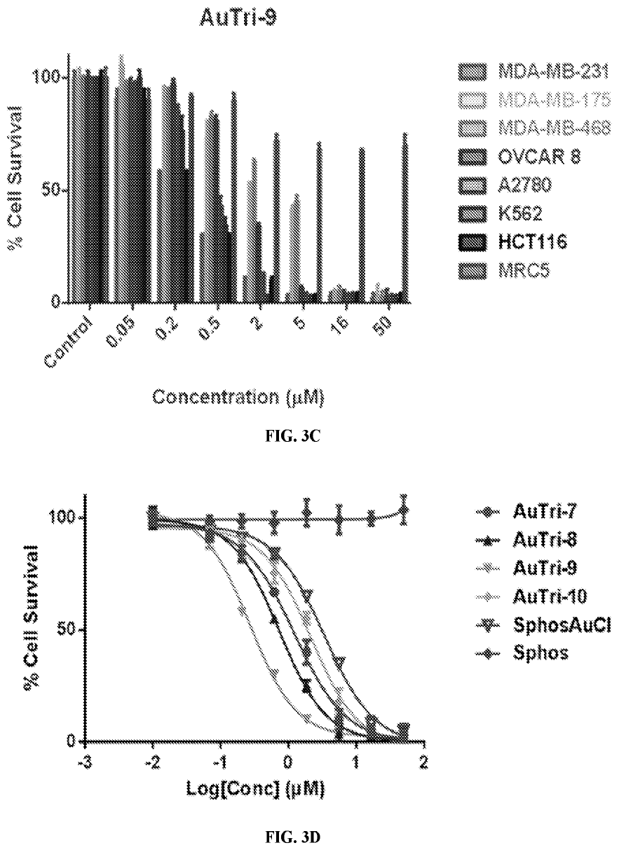

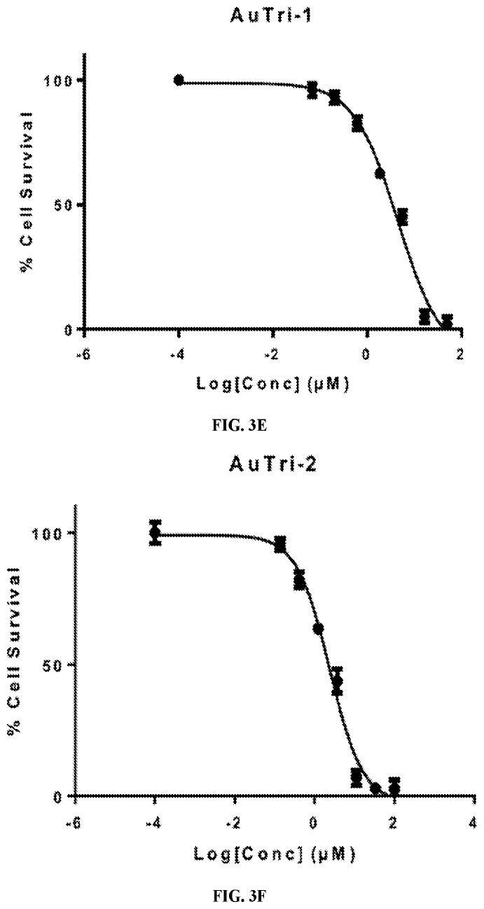

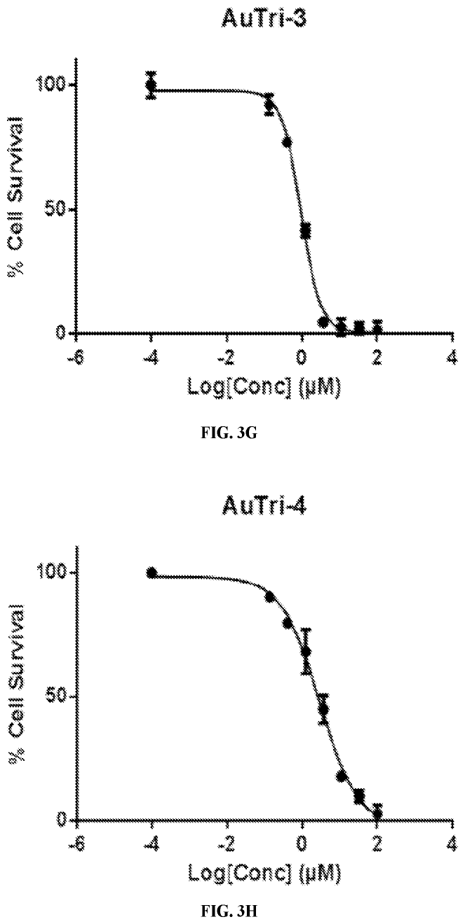

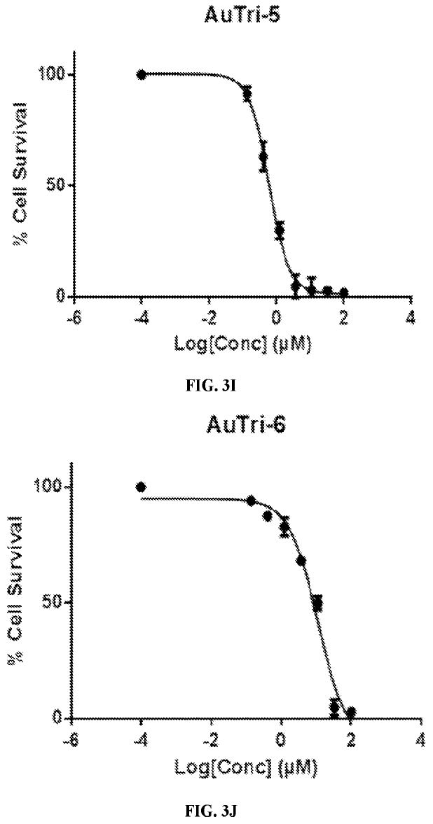

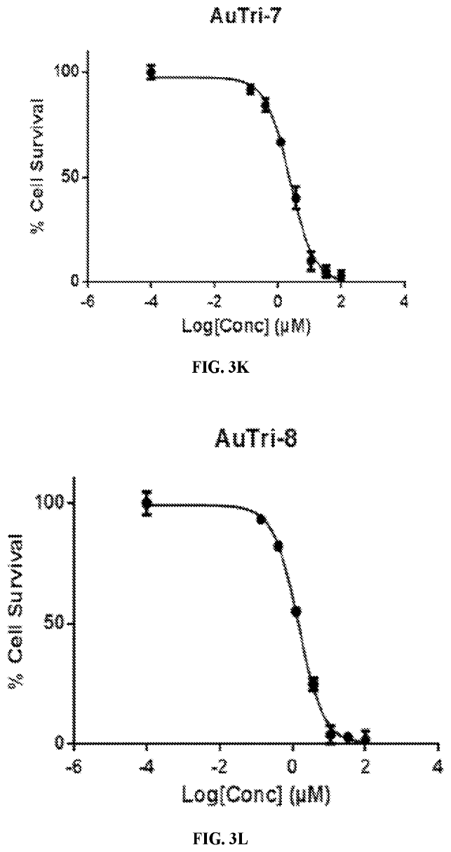

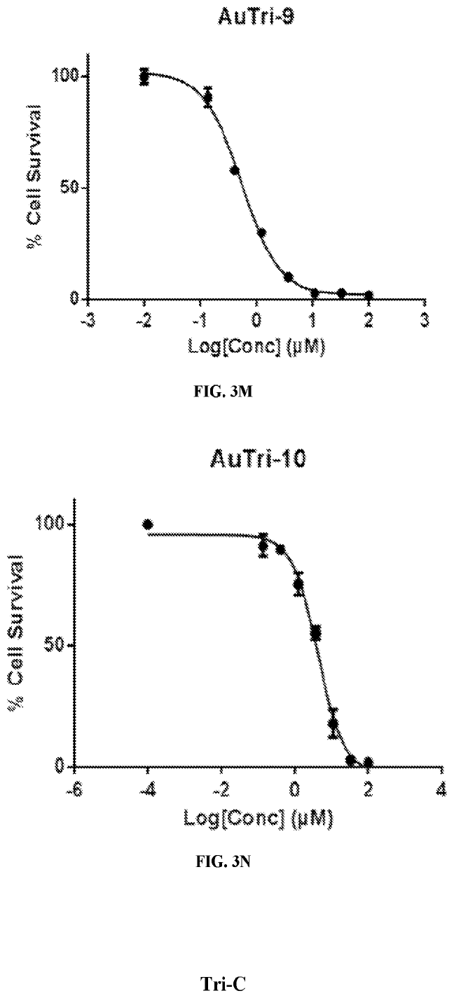

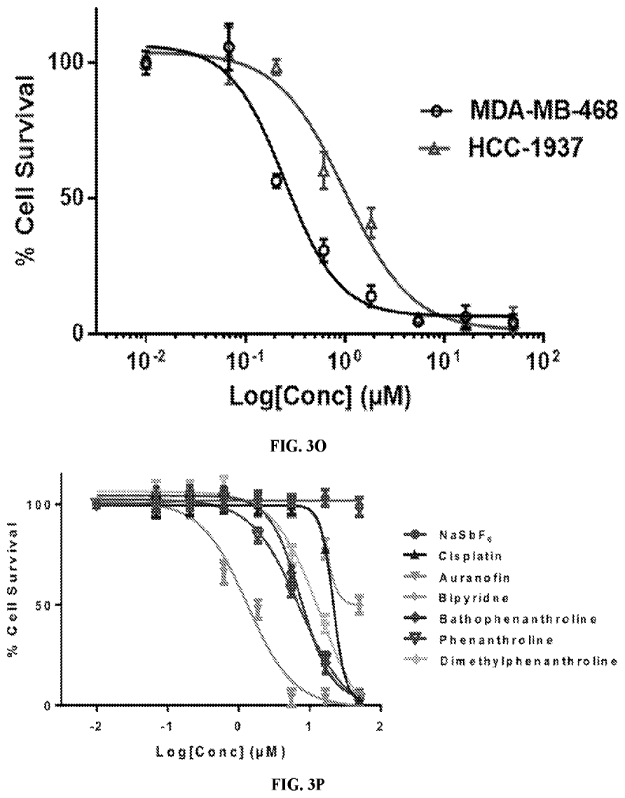

All the compounds were more cytotoxic in MDA-MB-231 cells in comparison to cisplatin (IC₅₀=34.56±0.783 μM, Table 1). Next, cellular selectivity was evaluated by testing the ten complexes in MRCS, a normal lung fibroblast cell line (FIG. 3A). Interestingly, these complexes all exhibited IC₅₀ values over 50 μM. This selectivity is key, as toxicity to healthy cells is a main source of harmful side effects with regards to traditional platinum based chemotherapeutics. Overall, complexes bearing the Sphos ancillary ligand (i.e. AuTri-7-10) were more effective than PPh₃ and AsPh₃. To establish that the observed cytotoxicity was due to the gold(I) complex itself and not the bidentate framework, all four bidentate ligands were evaluated in MDA-MB-231.

The cytotoxicity of the ligands were >7 μM (Table 1), indicating that the observed cytotoxicity of the AuTri complexes is not attributed solely to the bidentate backbone. Next, the effect of the SbF₆⁻ counterion was evaluated. Common counterions used in biologically relevant transition metal complexes are BF₄⁻ and the most common being PF₆⁻. NaSbF₆ was used as the source of the counterion, and there was no cytotoxicity observed (IC₅₀>50 μM, Table 1). Furthermore, structure activity relationships between the different bidentate ligands revealed that the derivatives of bathophenanthroline (bphen) were more toxic than their counterparts (FIG. 3B). This is attributed to improved stability and lipophilic character of the bphen ligand. The combined SAR identified AuTri-9 as a promising candidate, having an IC₅₀ value of 0.54±0.03 μM (Table 1). To evaluate the efficacy of AuTri-9 further, it was compared to known FDA approved drug, auranofin. Given the observed IC₅₀ value of auranofin (1.31±0.15 μM, Table 1), AuTri-9 proved to be a potent candidate for extensive biological characterization. Next, the efficacy of AuTri-9 was explored in an array of cell lines in an attempt to validate the effect across a broad range of cell lines (FIG. 3C). AuTri-9 demonstrated competitive cellular toxicity across a panel of cell lines. Additionally, a comparison of the free ligand (Sphos) and gold(I) precursor (ClAuSphos), reveal that the observed cellular toxicity is a result of the unified AuTri complexes and not the ligands (FIG. 3D). Full sigmoidal plots of AuTri-1-10, Tri-C, phenanthroline, bathophenanthroline, bipyridine, dimethylphenanthroline, auranofin, cisplatin, and NaSbF₆ in MDA-MB-231 can be found in FIGS. 3E-3P.

### Example 4: Solution Stability and Reactivity

Solution Stability of AuTri-9. All media were warmed to 37° C. prior to use. DMEM was supplemented with 10% FBS, 1% amphotericin and 1% penicillin/streptomycin, PBS (1×) was purchased from Fischer Scientific and used as it, and DMSO was purchased as ACS grade was purchased from Sigma Aldrich and used as is. All absorption spectra were recorded on a Shimadzu UV-1280 model instrument. Prior to each run, the instrument was blanked with the corresponding buffer/solvent. The solutions were incubated at 37° C. until used for absorption measurement. For DMEM, AuTri-1-10 were prepared as a 1 mM stock in DMSO and diluted down to 50 μM with the corresponding medium. No precipitation was observed. The absorption spectra were recorded at each listed time interval. For DMSO and PBS, AuTri-9 was prepared as a 1 mM stock in DMSO and diluted to 50 μM. No precipitation was observed. The absorption spectra were recorded at each listed time interval.

Reactivity of AuTri-9 with NAC and GSH. All media were warmed to 37° C. prior to use. DMEM was supplemented with 10% FBS, 1% amphotericin and 1% penicillin/streptomycin, PBS (1×) was purchased from Fischer Scientific and used as it, and DMSO was purchased as ACS grade was purchased from Sigma Aldrich and used as is. All absorption spectra were recorded on a Shimadzu UV-1280 model instrument. AuTri-9 was prepared as a 1 mM stock in DMSO and diluted to 100 μM. Both NAC and GSH were dissolved in PBS (1 mM) and diluted to 100 μM with PBS. The solutions were added in the stated stoichiometric amounts and the absorption spectra recorded after 1 hour of incubation.

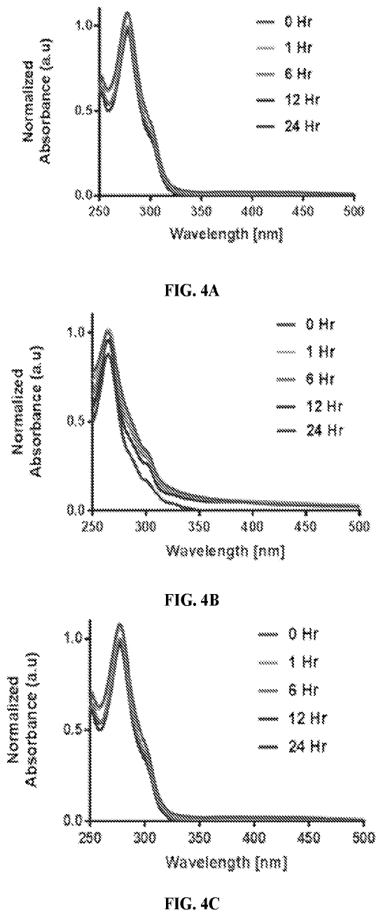

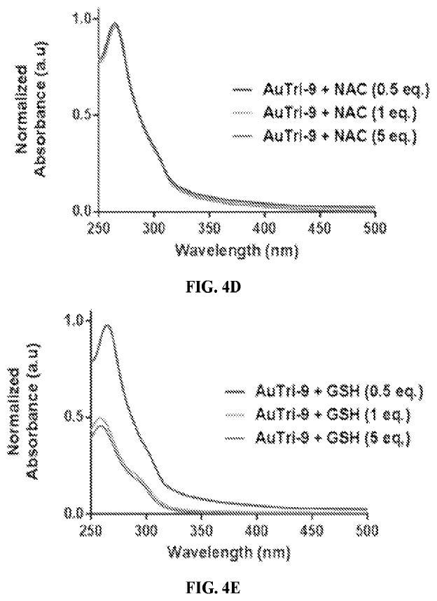

Solution Stability and Reactivity. Solution stability of metal complexes is an important factor in the development of effective transition metal based therapeutics. The longevity of the complex in biological systems determines important parameters including but not limited to cytotoxicity, maximum tolerated doses, and pharmacokinetics. To assess the stability of these gold complexes, they were initially subjected to time dependent stability studies in the biologically relevant media Dubellco modified essential medium (DMEM), see FIG. 4A for AuTri-9; data not shown for AuTri-1-8 and -10).

Changes in the absorption spectra of AuTri-1-10 were monitored over the course of a 24-hour time period, incubating the solutions at 37° C. PPh₃ derivatives were relatively stable over the 24-hour period, with the exception of the bpy analogue (AuTri-3). The extra electronics of the more conjugated phen and bphen derivatives provide enhanced stability to the gold metal center. Not surprisingly, the AsPh₃ analogues were not stable, rapidly reducing to elemental gold after the 12-hour mark. The increased bond length observed between Au-As in complexes AuTri-4-6 confirms the lability of those compounds and may explain their relatively rapid reduction. Complexes AuTri-7-10 proved to be very stable over the 24-hour period due to the stronger Au-P bonds. AuTri-9 was exceptionally stable over the 24-hour time period (FIG. 4A) with no alterations to the UV-vis spectra.

The solubility and stability of AuTri-9 was further evaluated in DMSO (FIG. 4B) and PBS (FIG. 4C) using UV-Vis spectroscopy. All complexes are readily soluble in both DMSO and MeCN, however; the stability of AuTri-9 was additionally evaluated in DMSO and PBS solutions over an extended time period. When evaluated over a 24 hour time period, the complex exhibits minimal changes in its absorption profile, indicative of suitable stability and solubility of the compound in solution.

Further, the reactivity of AuTri-9 was explored with common thiol reductants including N-acetyl cysteine (NAC) and L-glutathione (GSH). The reaction was monitored by UV-Vis spectroscopy. A PBS solution of AuTri-9 was incubated with both NAC (FIG. 4D) or L-GSH (FIG. 4E) at varying equivalents over 1 hour. The reaction involving NAC did not impact the absorption band of AuTri-9, suggestive of no reaction with NAC. Conversely, when AuTri-9 was exposed to L-GSH, a decrease in the absorbance spectrum was observed, indicating reactivity with this nucleophile. Given that intracellular thiols are common binding targets of gold(I) complexes, this interaction is not surprising. Of note, there was no reduction to elemental gold.

### Example 5: Mitochondrial Morphology and Structure

Transmission Electron Microscopy. MDA-MB-231 cells were seeded in 6 well plates and allowed to grow to 85% confluency. The cells were then treated with AuTri-9 at a concentration of 10 μM (<1% DMSO) for 1 h at 37° C. Growth media were removed and immediately and added 3% glutaraldehyde at 4° C. in 0.1 M Sorenson's or Cacodylate Buffer. After 5 minutes remove fix and replace with fresh fixative, leave for 45 min-1 hr at 4° C., washed in 0.1 M buffer w/5% sucrose 4×, 5 minutes each. Post fix, the cells were stained with 1% OₛO₄ in 0.1 M buffer for 1 hr at 4° C. and washed in 0.1 M buffer 1×. The cells were dehydrated in graded ethanols, 5 minutes each, from 50% through 100% and absolute ethanol (2×) at 4° C. After the last alcohol wash remove the residual alcohol with a small pipette and immediately add straight resin (14.5 g Eponate 12, 8.0 g DDSA, 7.15 g NMA, and 0.85 gm BDMA) to the dish. Swirl the dish and then remove as much of the resin, (which may contain residual alcohol), with a plastic pipette being careful not to disturb the cell layer. Then add straight resin 2× for 1-2 hours and place under the lamp, on a shaker. Pour off the 2nd straight resin wash, let the plate drain well (leaving a layer of resin on the bottom) and invert labeled beem capsules into each well that have had the conical ends removed. Place the smooth factory end down in the wells being careful not to move the capsules around too much and disturb the cell layer and polymerize overnight at 60° C. The following morning fill the beem capsules with fresh resin and polymerize for another 24 hours at 60° C. With the aid of a metal chemical spatula snap the capsules off the dishes when cool. The capsules were then sectioned using a fresh microtome. Transmission electron microscopy (TEM) and scanning transmission electron microscopy (STEM) were performed on a Thermo Scientific Talos F200× microscope, operated at an accelerating voltage of 200 kV or/and 80 kV. Velox digital micrograph software was used to record TEM/STEM images, measure cell size, and measure changes in mitochondrial morphology.

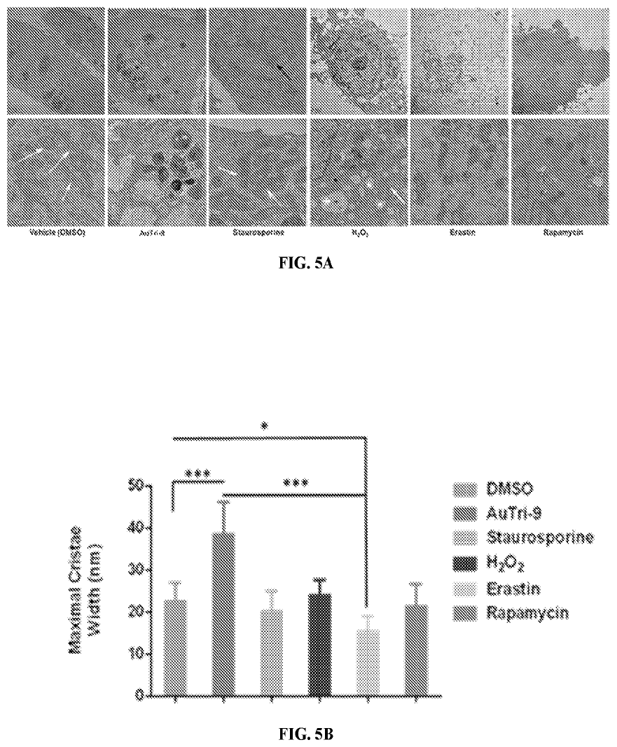

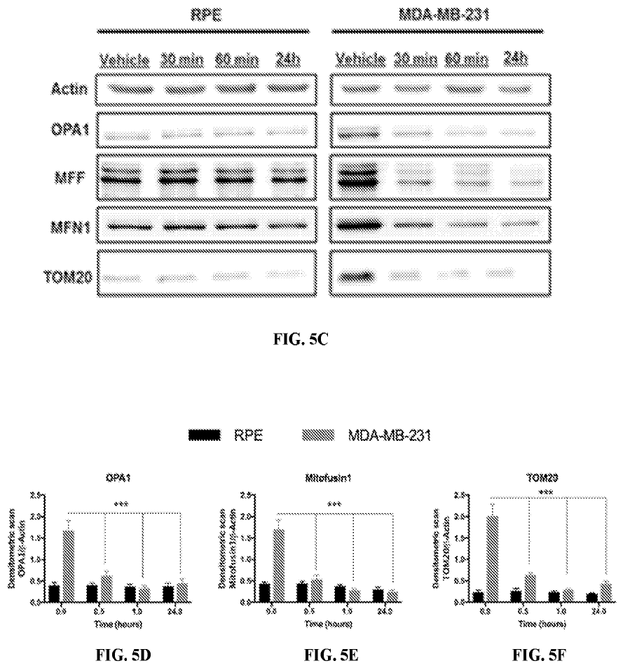

Compound AuTri-9 Directly Affects Mitochondrial Morphology and Structure. To examine the direct effect of the geometrically distinct gold agents on mitochondria AuTri-9 was selected. Morphological changes to mitochondria are associated with different cell phenotypes or cell death mechanisms. Targeting mitochondrial processes is a fruitful arena for metallodrug discovery.^{36-37, 46-48} Transmission electron microscopy (TEM) was used to look at the exact morphological changes in mitochondria of cells exposed to AuTri-9. MDA-MB-231 were treated with AuTri-9, staurosporine (STS) an apoptosis inducer (e.g., chromatin condensation and margination); hydrogen peroxide, which induces necrosis (e.g., cytoplasmic and organelle swelling, plasma membrane rupture); rapamycin for cellular induced autophagy (e.g., formation of double-membrane enclosed vesicles) and erastin, a known ferroptosis inducer (FIG. 5A).⁴⁹

When comparing AuTri-9 with vehicle control, clear rupture of mitochondrial membranes can be observed along with fragmented cristae (gray arrow). Healthy mitochondria (white arrow) show distinct intact outer membrane and cristae, thus the compound appears to be directly effecting the structure of mitochondria. Comparing AuTri-9 treatment with H₂O₂, the distinct cytoplasmic swelling and membrane rupture (white star) is observed; however, the mitochondria structure remains intact. Comparison to erastin showed opposite effects, AuTri-9 demonstrated fragmentation and destruction of mitochondria structure, whereas erastin showed mitochondrial shrinkage (arrow). Furthermore, comparison of mitochondrial features with both rapamycin and staurosporine revealed dissimilar morphological changes. Both rapamycin (white arrow) and staurosporine (grey arrow) showed no distinct morphological changes to mitochondria.

Mitochondrial cristae play a pivotal role in overall cell health. Under normal biological conditions, mitochondria undergo fusion/fission and cristae form junctions throughout the mitochondria. This allows for the redistribution of cytochrome C from the cristae lumen to the intermembrane space.⁵⁰ As earlier noted, mitochondrial bioenergetics and normal mitochondrial structure are crucial to maintaining MMP, the driving force of ATP production. Using Velox digital micrograph software, the cristae width was measured within each treatment condition to quantify and compare the morphological change (FIG. 5B). Treatment with AuTri-9 resulted in significantly increased maximal cristae width in comparison to the vehicle control, suggesting assembly of functional mitochondrial respiratory chain complexes and efficiency of mitochondria-dependent cell growth. This phenomenon may be exploited to develop therapeutics and study mitochondrial dynamics. Overall, a distinct profile to mitochondrial structure disruption can be achieved in a rapid manner using AuTri-9.

Immunoblotting. To investigate the consequence of mitochondrial structure perturbation, immunoblotting was performed for critical proteins involved in maintaining mitochondrial structure homeostasis. Equal numbers of RPE and MDA-MD-231 cells were seeded and treated with AuTri-9 for the indicated time points. Whole cell lysates were prepared using RIPA buffer, 1× protease inhibitor cocktail (Sigma), and 1× phosphatase inhibitor cocktails I and II (Sigma) and loaded by equal protein for SDS-PAGE. Protein concentrations in the cell lysates were determined with the Bradford protein assay reagent. Cell lysates containing equal amounts of protein were separated on a 4-20% SDS-polyacrylamide gel. Post separation, proteins were transferred to a nitrocellulose membrane and non-specific binding sites were blocked by treating with 5% nonfat dry milk. The membranes were incubated overnight with the primary antibodies directed against OPA1, MFF, MFN1, TOM20, SOD1, SOD2, NRF2, cytochrome c, and beta-actin. Appropriate secondary antibodies were used accordingly. Blots were visualized with Thermo Scientific Pierce Supersignal West Dura Extended Duration Chemiluminescent Substrate on the LI-COR C-DiGit Chemiluminescent Western Blot Scanner (Lincoln, NE). Measurement of integrated density of protein bands was performed using ImageStudioLite software (LI-COR).

TOM20, a protein located on the outer membrane as a component of the import receptor complex, MFN1, a key protein responsible for mitochondria fission, MFF, a protein primarily responsible for mitochondrial division, and OPA1, a critical protein involved in maintaining mitochondrial structure, were all examined (FIG. 5C).⁵¹⁻⁵⁵ Immunoblotting in MDA-MB-231 as well as the normal cell model, RPE, revealed a decrease in protein content of all four structure proteins in MDA-MB-231. Notably, there was no change in protein levels in the healthy cell model.

Normalized protein levels confirmed the noted changes (FIGS. 5D-5F). Repeated measures ANOVA found significant effect of cell type, AuTri-9 exposure time, and an interaction between cell type and AuTri-9 exposure time for OPA1, TOM20, and MFN1. It is worth noting that rapid protein changes were observed within 30 minutes of treatment. The downregulation of OPA1 by immunoblotting and quantitative proteomics, suggests that this protein is a potential direct target of AuTri-9. Recently, a study demonstrated that in HeLa cells, loss of OPA1 leads to fragmentation of the mitochondrial network in conjunction with loss of mitochondrial membrane potential thus causing fragmentation and disorganization of mitochondrial cristae which resulted in caspase-dependent apoptotic death. With this knowledge, it can be stated that disruption of mitochondrial structure is just as pertinent to cell homeostasis as any other biological system.

### Example 6: Intracellular Reactive Oxygen Species (ROS)

ROS Accumulation using DCF-DA. DCF-DA was purchased from Cayman Chemicals, stored at −20° C., and used without light. DCF-DA was prepared as a 1 mM stock in DMSO and diluted to a working concentration of 20 μM with PBS. AuTri-9 was prepared as a 1 mM stock in DMSO and diluted to a working concentration of 25 μM with PBS. MDA-MB-231 cells were grown to 85% confluency at 37° C. with 5-10% CO2. The cells were trypsinized, washed with PBS, and aliquoted to a density of 3×10⁵ cells, centrifuged, and suspended in 200 μL of a 20 μM DCF-DA solution prepared above. The cell suspensions were incubated at 37° C. for 30 minutes. AuTri-9 was added (50 μL) to achieve a final volume 250 μL and a final concentration of 5μM of AuTri-9 and analyzed using FACS (FITC channel, excitation 488 nm) at the given time intervals. After 4 hours, no response was observed as the population were dead (>90%). Tert-butyl hydroperoxide was used as the positive control. Data were analyzed using BD CellQuest Pro and plotted using GraphPad Prism 6.

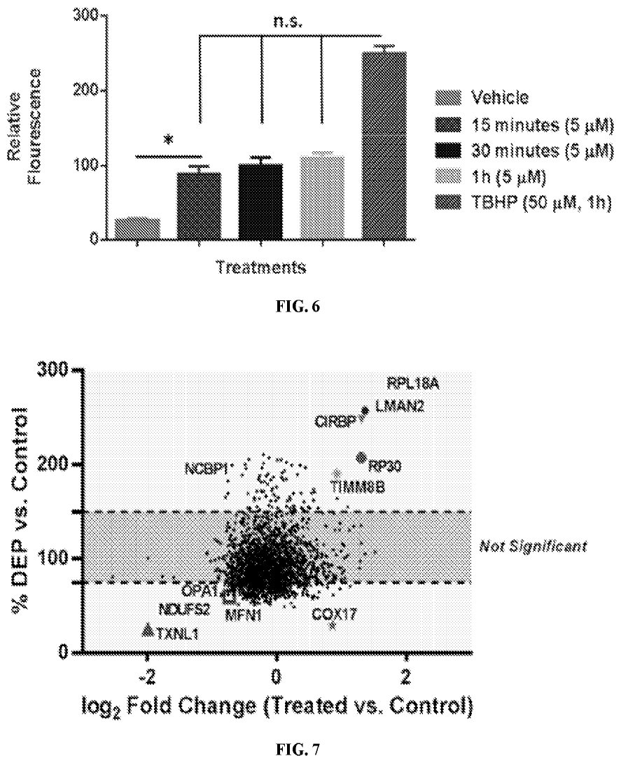

Compound AuTri-9 Generates Intracellular ROS. Given the recent reports of gold based chemotherapeutics inducing apoptosis as a result of ROS generation, the ability of AuTri-9 to generate intracellular ROS was investigated. ^{36, 56-60} Using 2′, 7′-dichlorofluorescin diacetate (DCF-DA) a fluorogenic dye which has its fluorescence activated upon oxidation via ROS species. After cell staining, MDA-MB-231 cells were subjected to AuTri-9 treatment at 5 μM and ROS levels measured using flow cytometry (ex. FITC channel) at different time points (15 minutes, 30 minutes, and 1 hour) (FIG. 6). Tert- butyl hydroperoxide was used as a positive control (50 μM, 1 hour). After a 15-minute treatment time, significant increase in ROS levels was observed in comparison to the untreated control group. However, after 30 minutes, and furthermore at 1 hour, there was no difference in the ROS increase observed earlier for the 15 minute treatment. The phenomenon is attributed to initial oxidative stress on the cells upon exposure to AuTri-9. However, since ROS levels do not drastically increase over exposure time, it is possible that ROS may not be a major contributor to cell cytotoxicity.

### Example 7: Quantitative Proteomics

MDA-MB-231 cells were seeded on petri dish (100 mm×15 mm) and allowed to grow to 85% confluency. The cells were then treated with AuTri-9 at a concentration of 1μM for 12 h at 37° C. Cells were harvested and sent to Creative Proteomics (Shirley, NY). Chemicals and Instrumentation used are as follows: TMT10plex Isobaric Label Reagent Set, Pierce Quantitative Colorimetric Peptide Assay, was purchased from Thermo Fisher Science. Triethylammonium bicarbonate buffer (1.0 M, pH 8.5±0.1), Tris(2-carboxyethyl)phosphine hydrochloride solution (0.5 M, pH 7.0), iodoacetamide (IAA), formic acid (FA), acetonitrile (ACN), methanol, were purchased from Sigma (St. Louis, MO, USA). Trypsin from bovine pancreas was purchased from Promega (Madison, WI, USA). Ultrapure water was prepared from a Millipore purification system (Billerica, MA, USA). An Ultimate 3000 nano UHPLC system (Thermo Scientific, Waltham, MA) coupled online to a Q Exactive HF mass spectrometer (Thermo Scientific) equipped with a Nanospray Flex Ion Source (Thermo Scientific). Cell pellets were lysed by using 200 μl of RIPA lysis buffer including protease inhibitors, centrifuged at 12000 rpm for 15 min at 4° C. and transfer the supernatant to a new EP tube and protein concentration determined by using BCA kit. Transfer 200 μL sample into a new microcentrifuge tube.

To each sample tube, reduced by 10 mM TCEP at 56° C. for 1 h, then alkylated by 20 mM IAA at room temperature in dark for lh. Next, samples were added free trypsin into the protein solution at a ratio of 1:50, and the solution was incubated at 37° C. overnight and lyophilized the extracted peptides to near dryness. Re-dissolve the sample with 100 mM TEAB. Samples were then labelled with peptide and fractionated. Samples were then analyzed by Nano LC-MS/MS. Nanoflow UPLC: Ultimate 3000 nano UHPLC system (ThermoFisher Scientific, USA); Nanocolumn : trapping column (PepMap C18, 100Å, 100 μm×2 cm, 5 μm) and an analytical column (PepMap C18, 100Å, 75 μμm×50 cm, 211.m);

Loaded sample volume: 5μL Mobile phase: A: 0.1% formic acid in water; B: 0.1% formic acid in 80% acetonitrile. Total flow rate : 250 nL/min LC linear gradient: a linear gradient from 5 to 7% buffer B in 2 min, from 7% to 20% buffer B in 80 min, from 20% to 40% buffer B in 35 min, then from 40% to 90% buffer B in 4 min. Q Exactive HF mass spectrometer (Thermo Fisher Scientific, USA) Spray voltage: 2.2 kV Capillary temperature: 270° C. MS parameters: MS resolution: 120000 at 200 m/z MS precursor m/z range: 300.0-1650.0 www.creative-proteomics.com 45-1 Ramsey Road, Shirley, NY 11967, USA Tel:1-631-275-3058 Fax:1-631-614-7828 www.creative-proteomics.com info@creative-proteomics.com MS/MS parameters: Product ion scan range: start from m/z 100 Activation Type: CID Min. Signal Required: 1500.0 Isolation Width: 3.00 Normalized Coll. Energy: 40.0 Default Charge State: 6 Activation Q: 0.250 Activation Time: 30.000 Data dependent MS/MS: up to top 15 most intense peptide ions from the preview scan in the Orbitrap

Quantitative Proteomics. Considering previous effects of gold molecules on intracellular thiols, to use quantitative proteomics was sought to gain a deeper understanding of the potential targets or pathways impacted by AuTri-9. AuTri-9-treated MDA-MB-231 cells were subjected to TMT-labeled quantitative proteomics and analyzed for differential protein changes compared to vehicle-treated MDA-MB-231 cells. The raw MS files were analyzed and searched against HUMAN protein database based on the species of the samples using Maxquant. Only high confident identified peptides were chosen for downstream protein identification analysis. A quantitative ratio over 1.5 (150% with respect to the control) was considered upregulation while quantitative ratio less than 1/1.5 (67% with respect to the control) was considered as down-regulation. The data revealed significant down regulation of RAS family proteins, specifically GTPases and RAB proteins. More specifically, AuTri-9 downregulated mitochondria structural proteins (e.g. OPA1, MFN1, and TOM20) without affecting protein content of the OXPHOS machinery and cell metabolism based proteins (including SOD1, SOD2, and NRF2) (FIG. 7). Other proteins of interest were also found to be downregulated, such as TXLN-1 (FIG. 7), a sulfur containing thioredoxin like protein. Unsurprisingly, downregulation of these proteins is common given the affinity of a soft polarizable metal like gold and the softer nature of sulfur atoms. However; after compilation of significant protein concentration changes, the findings indicated that these compounds could potentially be attacking mitochondria structure rather than cell metabolism. Together, protein studies corroborate the rapid disruption of mitochondrial structure induced by AuTri-9.

### Example 8: Cellular Metabolism with Treatment

Mitochondrial Membrane Potential (JC-1). MDA-MB-231 cells were plated at a density of 5×10⁵ cells/plate using a glass bottom petri dish fitted with a #1.5 cover slip with a final volume of 1.5 mL and allowed to adhere overnight at 37° C. Compound AuTri-9 was prepared as a stock in DMSO and added at a final concentration of 10 μM (<1% DMSO). The cells were treated for 1 h at this concentration. CCCP was prepared as a stock in DMSO and added at a final concentration of 5 μM and the cells treated for 15 minutes. This was used as a positive control. After the indicated treatment time, a working solution of the JC-1 dye (Cayman Chemicals) was prepared by adding 100 μL of dye into 900 uL of DMEM. Note: the working solution of JC-1 should always be prepared fresh and not stored for long-term use. Then, 100 μL/mL of DMEM were added to the cells and incubated at 37° C. for 20 minutes. Prior to imaging, the media was removed and replaced with room temperature PBS (2 mL). The cells were then visualized using confocal microscopy on a Nikon MR Inverted Confocal Microscope. J-aggregates were imaged with (excitation/emission: 510/590 nm) and J-monomers with (excitation/emission: 488/525 nm).

Mitochondrial Metabolism Analysis with Seahorse XF96 Analysis. The optimum conditions for cell density and FCCP injection concentration were determined to be 30,000 cells/well and an FCCP injection concentration of 0.6 μM.¹⁵ All Seahorse XF96 experiments with MDA-MB-231 were performed under these conditions. The cells were seeded the night prior to the experiment with a final volume of 100 uL and incubated overnight at 37° C. Compound AuTri-9 was prepared as a stock in DMSO and diluted to a working concentration of 200 μM with Seahorse XF96 assay buffer and then subsequently serial diluted by 3× to achieve multiple concentrations. The assay was performed using a pneumatic injection method of AuTri-9, with the final injection concentrations of 0.1, 1, and 3 μM (<1% DMSO). This was followed by injection of oligomycin (1.5 μM), FCCP (0.6 μM) and rotenone/ antimycin A (0.5 μM). The metabolic parameters are calculated as previously reported. 1617

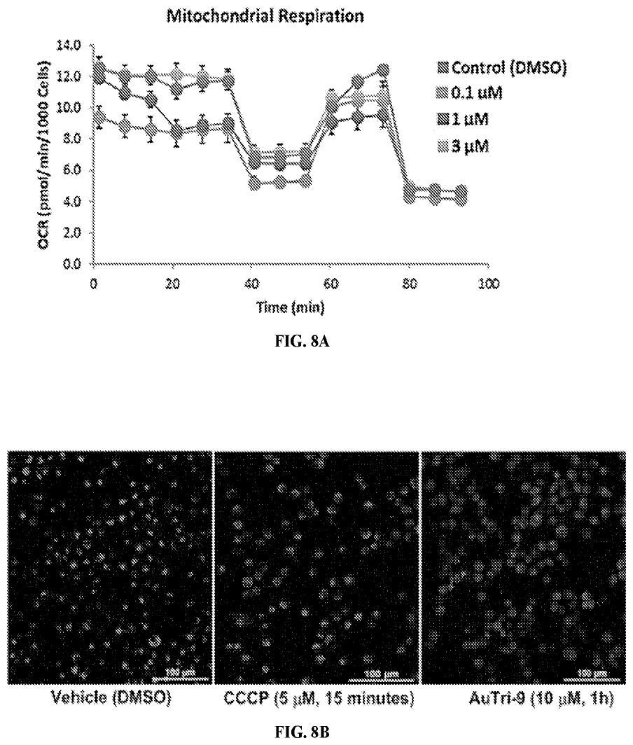

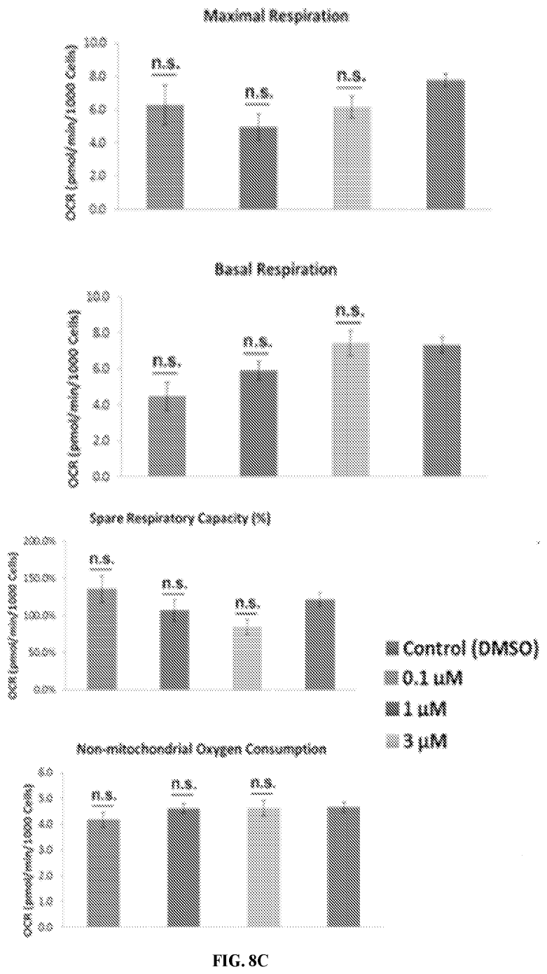

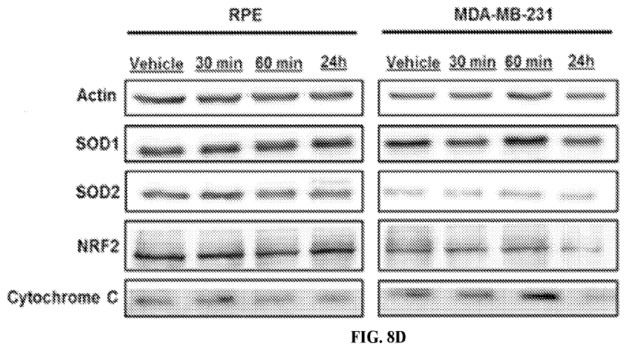

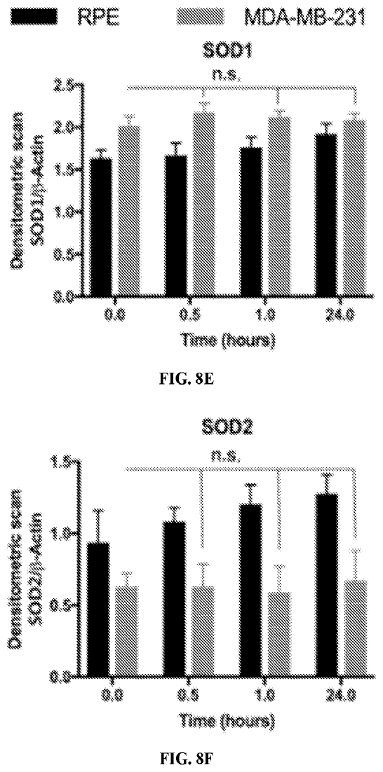

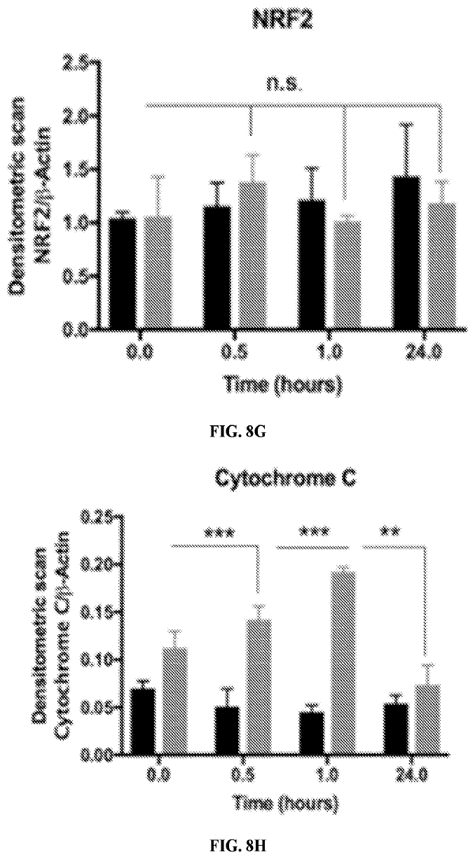

Cellular Metabolism Remains Unaffected Upon Treatment with AuTri-9. To fully understand the mechanism of action, knowing that mitochondrial based proteins were modulated, the effect of AuTri-9 treatment on metabolism was analyzed. Compounds that disrupt the electron transport chain (ETC) are potent inhibitors of cell metastasis as cancer cells hijack OXPHOS machinery to support growth.⁶¹ First, cells were treated with AuTri-9 via pneumatic injection in a mitostress assay using Seahorse XF96 and analyzed the changes in OCR (oxygen consumption rate) over time. Next, known OXPHOS inhibitors were injected: oligomycin, a complex V inhibitor, to view the basal OCR; FCCP, an uncoupler used to observe the maximum OCR, and rotenone/antimycin A, a complex I/III inhibitor to completely shut down the ETC (FIG. 8A). Surprisingly, at concentrations 6 times the IC₅₀ of AuTri-9, no significant change in OCR was observed after the 90-min experimental protocol. Often, pneumatic injection is preferred over pretreating cells with the agent under investigation to better recapitulate direct metabolic effects instead of cell-death associated events. For example, Gboxin, a known small molecule inhibitor of OXPHOS, induces rapid and irreversible inhibition of overall oxygen consumption rate to demonstrate specificity for mitochondrial respiration. 62 Further evaluation of key metabolic parameters including basal oxygen consumption rate, maximal oxygen consumption rate, spare respiratory capacity, and non-mitochondrial dependent oxygen consumption remain unaltered (FIG. 8B). Although OXPHOS is critical for cancer cell proliferation and survival, it is clear that other cell pathways involving mitochondria can be a target for cell death. Next,MDA-MB-231 cells were treated with AuTri-9 to analyze the mitochondrial membrane polarization, using a known uncoupler CCCP as a positive control (FIG. 8C). The mitochondrial specific dye JC-1 was used, where green fluorescence represents J-monomers and red fluorescence represents J-aggregates.⁶³ Interestingly, a stark change in MMP was observed on treatment in 1 hour. This drastic change in MMP suggests that the structural changes observed contribute to depolarization. Then immunoblotting of key proteins involved in the OXPHOS machinery was performed. These included SOD1, SOD2, NRF2, and Cytochrome C (FIG. 8D). These proteins were chosen as SOD1/50D2 inhibition induces cell death by diminishing antioxidant protein activities, leading to an increase in H₂O₂ as they are a key component in reducing intracellular ROS. Increased NRF2 activity provides defense against mitochondrial toxins. Furthermore, down regulation of NRF2 is found in several mitochondrial based diseases such as Parkinson's disease.⁶⁴ Finally, Cytochrome C levels were evaluated as it is released into the cell when a cell receives apoptotic stimuli. Given the key roles these proteins play in maintaining redox homeostasis, immunoblotting provides concrete evidence of protein level upon treatment. In contrast, upon treatment of both RPE cells and MDA-MB-231 cells, no change in protein levels could be observed. Measurements of integrated density of protein bands was performed using Image Studio Software (Version 5.2.5), with background correction calculated using a signal ratio error model. To quantify the protein content (FIGS. 8E-8H), the bands were analyzed with LI-COR and normalized by β-actin levels. Calculations of relative signal were normalized to untreated sample for each set, as indicated. Analysis of the immunoblots showed no significant changes in the proteins examined, suggesting that AuTri-9 does not interact directly with OXPHOS machinery or key cytosolic proteins that maintain cell redox homeostasis.

### Example 9: Uptake Mechanism

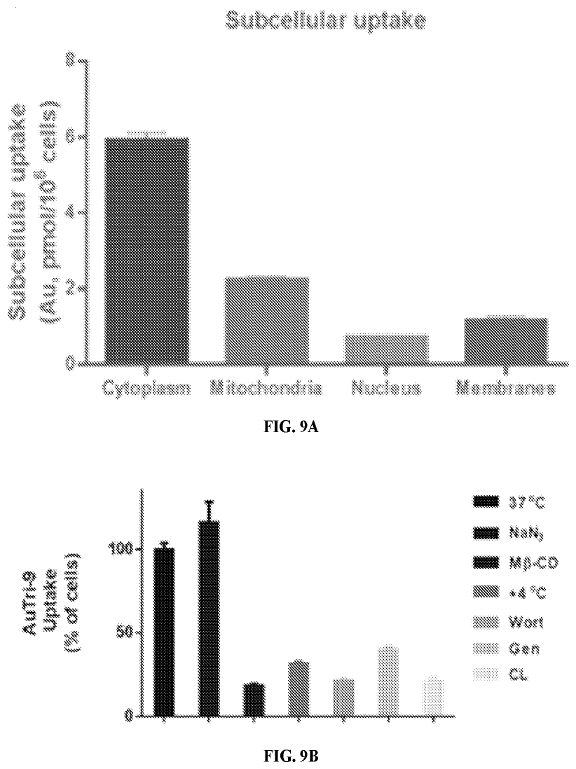

Cellular uptake of AuTri-9 and mechanism. The uptake mechanism of AuTri-9 in cells was explored. Understanding the target and uptake pathway is crucial when developing new therapeutic agents or probes. Subcellular localization of therapeutic agents to better understand specific target binding and the affinity a therapeutic has towards certain parts of the cell. This insight helps further elucidate the mechanism of action of therapeutic agents as one can further pinpoint the specific binding target. Gold content in specific cellular fractions was analyzed including, nucleus, cytoplasm, membranes, and mitochondria. This data revealed that localization of AuTri-9 was found to occur in the cytoplasm of the cell (FIG. 9A). This can be attributed to the lipophilicity associated with AuTri-9. Relatively lower gold uptake was observed in both the nucleus and cellular membranes. The gold compound localized appreciably within the mitochondria at >2 pmol/million cells. To determine the potential mechanism of cellular uptake, MDA-MB-231 were first treated with AuTri-9 to determine the whole cell uptake. Next, cells were pre-treated with known uptake inhibitors: sodium azide (NaN₃), which is known to inhibit mitochondrial oxidative phosphorylation and was used as a general inhibitor of energy (ATP)-dependent (active) uptake; chlorpromazine (CL) was utilized as an inhibitor of clathrindependent endocytosis (CDE); b-cyclodextrin (Me-β-CD) and genestein (Gen) were used as inhibitors of clathrin-independent endocytosis (CIE); and, wortmannin (Wort) was employed as a known inhibitor of micropinocytosis (FIG. 9B). The high cellular uptake of Au with respect to the control when pre-treated with NaN₃ shows that OXPHOS is not a key factor in uptake of the compound. This further confirms the hypothesis that these compounds do not directly affect OXPHOS machinery in cancer cells. Other inhibitors collectively show a reduction in Au uptake, indicative of a broad uptake mechanism. Overall, this study illustrates a new way of targeting mitochondria via structural perturbation, rather than direct interaction of OXPHOS machinery.

Whole Cellular Uptake of AuTri-9. For whole cellular uptake, MDA-MB-231 cells were seeded at a density of 1×10⁶ cells/mL in a 6 well clear bottom plate with a volume of 2.5 mL and allowed to adhere overnight at 37° C. Compounds were prepared as a stock in DMSO and added to each well at a final concentration of 1 μM (<1% DMSO) and treated for 6 h. The cells were then collected by trypsinization and centrifuged at 2000 rpm for 5 minutes to form a pellet. The pellet was suspended in 1 mL of DMEM, transferred to a 1.5 mL Eppendorf tube, and centrifuged again at 2000 rpm for 5 minutes. The media were removed, washed with PBS twice, and the pellet stored at −20° C. until analysis. Prior to analysis, the pellets were suspended in 0.5 mL of concentrated HNO3 and agitated for 1 minute. The solution was transferred to a 15 mL Falcon tube and then 4.5 mL of DI H2O was added. The samples were then subjected to analysis with ICP-OES. Data is represented as the mean ±s.e.m. (n=3).

Whole cellular uptake with pre-incubation of uptake inhibitors was performed as follows. MDA-MB-231 cells were seeded at a density of 1×10⁶ in clear-bottomed six-well plates and incubated at 37° C. overnight. The cells were then pre-treated with the following inhibitors: NaN3 (1 mM), methyl-β-cyclodextrin (5 mM), chlorpromazine hydrochloride (28 nM), wortmannin (50 nM), genistein (200 μM), for 1 hour. For the +4 ° C. sample, the cells were cooled to +4° C. prior to addition of AuTri-9. Following pre-treatment, the media were removed and washed with PBS (3×2 mL). The cells were then incubated with AuTri-9 (5 μM for 24 hours, <1% DMSO) at 37° C. and the +4° C. sample incubated at the same concentration at the indicated lower temperature. After treatment, the cells were collected both, the medium and tryspsinzed cells combined, centrifuged at 2000 rpm for 5 minutes to form a pellet. The pellet was suspended in 1 mL of DMEM, transferred to a 1.5 mL Eppendorf tube, and centrifuged again at 2000 rpm for 5 minutes. The media were removed, washed with PBS twice, and the pellet stored at −20° C. until analysis. Prior to analysis, the pellets were suspended in 300 μL of 70% HNO3. 100 μL aliquot of this stock was then diluted to 1 mL and subjected to GF-AAS. A calibration curve using Au in varying concentrations was performed.

Subcellular Uptake of AuTri-9. The subcellular uptake of AuTri-9 was conducted in a similar manner per the instructions from the RayBioâ Nuclear Extraction Kit Protocol (RayBiotech, Inc.). MDA-MB-231 cells were seeded at a density of 10×10⁶ in 100 mm petri dishes and incubated overnight at 37° C. AuTri-9 was added at 1 μM (<1% DMSO) for 6 hours. Once the fractions had been isolated, the concentrate was subjected to GF-AAS analysis. Prior to analysis, a calibration curve using Au in varying concentrations was performed. Data is represented as the mean ±s.e.m. (n=3).

Mitochondrial Uptake of AuTri-9. The localization of Au content in mitochondria were performed by using Mitochondria Isolation Kit for Cultured Cells from ThermoFisher Scientific™.MDA-MB-231 cells were plated at a density of 20×10⁶ in 100 mm petri dishes and incubated overnight at 37° C. AuTri-9 was added at a concentration of 1 μM (<1% DMSO) for 6 hours. Once the fractions had been isolated, the concentrate was subjected to GF-AAS analysis. Prior to analysis, a calibration curve using Au in varying concentrations was performed. Data is represented as the mean ±s.e.m. (n=3).

### Example 10: In Vivo Studies

In vivo maximum tolerated dose using AuTri-9. Six-week-old female Nu/Nu Nude mice were purchased from the Charles River Laboratories (Shrewsbury, MA) and quarantined for seven days. Animals were randomized into two treatment groups (n=3) and given either PBS or AuTri-9 at an equivalent dose of 10 mg/kg body weight through intraperitoneal injection (every other day, three-time per week, 7x). Mice were weighed every other day to monitor potential side effects and stress.

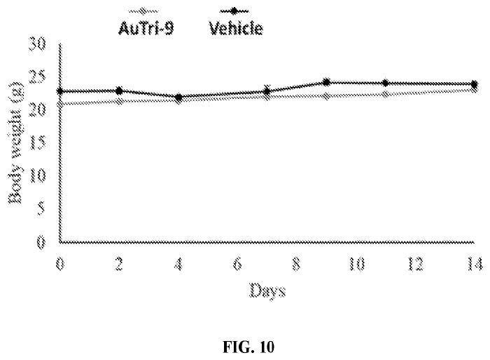

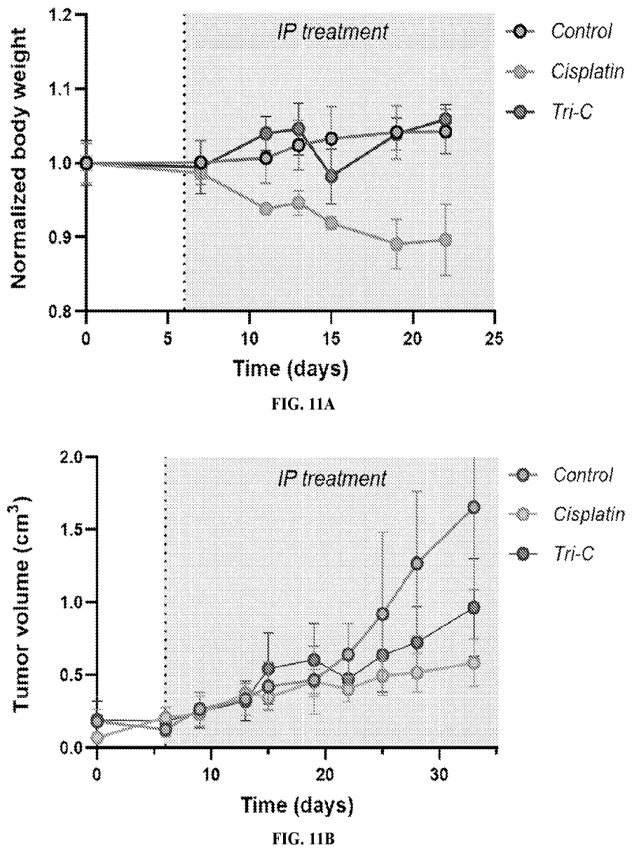

Preliminary In Vivo Studies of AuTri-9. To determine preliminary in vivo tolerance, female athymic Nu/nude mice were treated with AuTri-9 at a dose of 10 mg/kg based on gold (every other day, three-time per week, 7×). With known gold agents having a MTD (maximum tolerated dose from 3-10 mg/kg and auranofin (12 mg/kg based on gold), 10 mg/kg provided us with a more accurate estimation of overall in vivo toxicity. 65-68 The treatment group was compared to a separate control group of mice that were administered with 85% Kolliphor, 1% DMSO, and 4% PBS (n=2 mice per group) (FIG. 10). The weight and behavior of the mice were monitored every two days for signs of toxicity. After a 14-day treatment time, all mice were recorded with no significant change in body weight. This preliminary data suggests that the compound is tolerable in mice, a critical component in further drug/probe development

All together, the facile synthesis was leveraged to access a small library of unique tri-coordinate gold(I) complexes. This geometry appears to play a critical role in cellular toxicity due to the increase in toxicity over traditional linear gold(I) complexes of similar nature. These complexes exhibit high in vitro cytotoxicity with a clear improvement based on SAR. Furthermore, the compounds display a >35 times selectivity towards TNBC's over normal lung fibroblasts. AuTri-9 displayed promising cytotoxicity in multiple cell lines. With the use of quantitative proteomics, critical information was obtained, which signaled a mitochondrial driven mechanism. Combined bioenergetics studies revealed no significant effect on bioenergetics; however, a direct change in overall MMP, which suggests a change in mitochondrial dynamics independent from OXPHOS. Electron microscopy revealed significant morphological changes from traditional cell death pathways, which was further confirmed by immunoblotting of mitochondrial structure proteins. Cellular uptake was then quantified and revealed that AuTri-9 was taken up into the cell via a broad mechanism and gold content found to be localized in mitochondria. Finally, preliminary in vivo studies suggest that these compounds are potential candidates for further evaluation as AuTri-9 was tolerated in mice at 10 mg/kg over a two-week period. All of these factors illustrate for the first time, biological utility of tri-coordinate gold(I) complexes as mitochondrial probes and anticancer agents.

### Example 11: In Vivo Studies, Toxicity and Tumor Volume

BALB/c nude mice were injected subcutaneously into the flank region (dorsolateral region) with 3×10⁵ glioblastoma stem cells in 50 μL Matrigel. First measurements occurred at day 14 and treatment started 20 days after cell implantation, with 16.7 mg/kg body weight of metal complex (molar equivalent to 5 mg/kg of cisplatin) in a volume of 150 uL per mice, injected intraperitoneally (prepared from a stock solution of the metal complex at 10 mg/mL in DMSO, then diluted in 2% Tween 80 in PBS), and mice were then treated twice a week for 28 days. The first two weeks of the experiment related to tumor growth phase before imaging and treatment (data not shown). The control group was treated similarly with the vehicle. Fluorescence was monitored by the LifeScience Caliper software (v4.3), tumor volume was measured and calculated according to the following formula:

[symbol]

Volume(mm) [symbol] \hat{ }3)=([ [symbol] Width(mm) [symbol] \hat{ }2×Length(mm)])/2

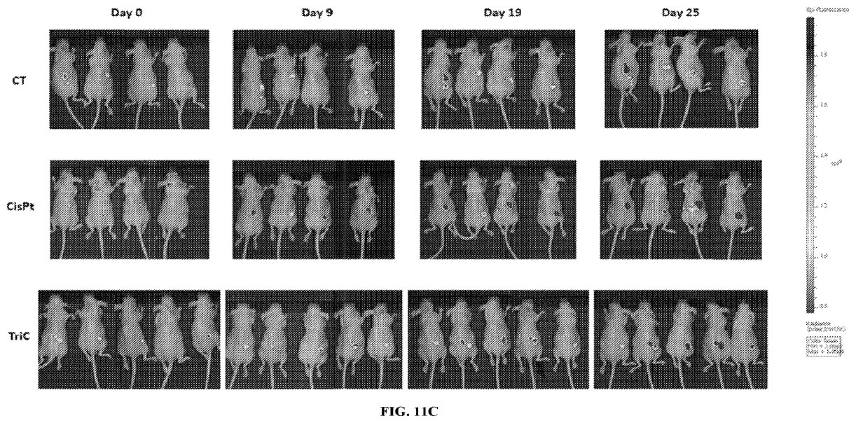

Normalized body weight as a function of time (FIG. 11A) and tumor volume as a function of time (FIG. 11B) are presented. Images of mice treated with Tri-C, Cisplatin, or vehicle control at Day 0, 9, 19, and 25 are provided (FIG. 11C).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Mannella, C. A., The relevance of mitochondrial membrane topology to mitochondrial function. Biochim. Biophys. Acta. 2006, 1762, 140-7.
2. Mishra, P.; Chan, D. C., Metabolic regulation of mitochondrial dynamics. J Cell Biol. 2016, 212, 379-387.
3. Mouli, P. K.; Twig, G.; Shirihai, O. S., Frequency and selectivity of mitochondrial fusion are key to its quality maintenance function. Biophys. 1 2009, 96, 3509-3518.
4. Westermann, B., Mitochondrial fusion and fission in cell life and death. Nat. Rev. Mol. Cel.l Biol. 2010, 11, 872-84.
5. van der Bliek, A. M.; Shen, Q.; Kawajiri, S., Mechanisms of Mitochondrial Fission and Fusion. Cold Spring Harbor Perspectives in Biology 2013, 5. a011072
6. Mertens, R. T.; Parkin, S.; Awuah, S. G., Cancer cell-selective modulation of mitochondrial respiration and metabolism by potent organogold(III) dithiocarbamates. Chem. Sci. 2020, 11, 10465-10482.
7. Chen, X.; Glytsou, C.; Zhou, H.; Narang, S.; Reyna, D. E.; Lopez, A.; Sakellaropoulos, T.; Gong, Y.; Kloetgen, A.; Yap, Y. S.; Wang, E.; Gavathiotis, E.; Tsirigos, A.; Tibes, R.; Aifantis, I., Targeting Mitochondrial Structure Sensitizes Acute Myeloid Leukemia to Venetoclax Treatment. Cancer Discov. 2019, 9, 890-909.
8. Finkelstein, A. E.; Walz, D. T.; Batista, V.; Mizraji, M.; Roisman, F.; Misher, A., Auranofin. New oral gold compound for treatment of rheumatoid arthritis. Ann. Rheum. Dis. 1976, 35, 251-257.
9. Nardon, C.; Boscutti, G.; Fregona, D., Beyond platinums: gold complexes as anticancer agents. Anticancer Res. 2014, 34, 487-92.
10. Kostova, I., Gold coordination complexes as anticancer agents. Anticancer Agents Med. Chem. 2006, 6, 19-32.
11. Kim, J. H.; Reeder, E.; Parkin, S.; Awuah, S. G., Gold(I/III)-Phosphine Complexes as Potent Antiproliferative Agents. Sci. Rep. 2019, 9, 12335.
12. Gukathasan, S.; Parkin, S.; Awuah, S. G., Cyclometalated Gold(III) Complexes Bearing DACH Ligands. Inorg. Chem. 2019, 58, 9326-9340.
13. Bertrand, B.; Casini, A., A golden future in medicinal inorganic chemistry: the promise of anticancer gold organometallic compounds. Dalton Trans. 2014, 43, 4209-4219.
14. Ndagi, U.; Mhlongo, N.; Soliman, M. E., Metal complexes in cancer therapy-an update from drug design perspective.Drug Des. Devel. Ther. 2017, 11, 599-616.
15. Marmol, I.; Quero, J.; Rodríguez-Yoldi, M. J.; Cerrada, E., Gold as a Possible Alternative to Platinum-Based Chemotherapy for Colon Cancer Treatment. Cancers (Basel) 2019, 11, 780.
16. Yue, S.; Luo, M.; Liu, H.; Wei, S., Recent Advances of Gold Compounds in Anticancer Immunity. Frontiers in Chemistry 2020, 8, 543.
17. Hu, D.; Lok, C.-N.; Che, C.-M., Anticancer Gold Compounds. In Metal-based Anticancer Agents, Cambridge, England: The Royal Society of Chemistry: 2019;120-142.
18. Elie, B. T.; Hubbard, K.; Pechenyy, Y.; Layek, B.; Prabha, S.; Contel, M., Preclinical evaluation of an unconventional ruthenium-gold-based chemotherapeutic: RANCE-1, in clear cell renal cell carcinoma. Cancer Medicine 2019, 8, 4304-4314.
19. Elie, B. T.; Hubbard, K.; Layek, B.; Yang, W. S.; Prabha, S.; Ramos, J. W.; Contel, M., Auranofin-Based Analogues Are Effective Against Clear Cell Renal Carcinoma In Vivo and Display No Significant Systemic Toxicity. ACS Pharmac. & Transl. Sci. 2020, 3, 644-654.
20. Gimeno, M. C.; Laguna, A., Gold chemistry with ferrocene derivatives as Ligands. Gold Bulletin 1999, 32, 90-95.
21. Gimeno M.C. Chemistry of Gold, Modern Supramolecular Gold Chemistry: Gold-Metal Interactions and Applications, Laguna, A. Ed., Weinheim, Germany: Wiley 2008.
22. Crespo, O.; Gimeno, M. C.; Laguna, A.; Jones, P. G., Two-, three- and four-co-ordinate gold(I) complexes of 1,2-bis(diphenylphosphino)-1,2-dicarba-closo-dodecaborane. J. e Chem. Soc., Dalton Trans. 1992, (10), 1601-1605.
23. Visbal, R.; Ospino, I.; López-de-Luzuriaga, J. M.; Laguna, A.; Gimeno, M. C., N-Heterocyclic Carbene Ligands as Modulators of Luminescence in Three-Coordinate Gold(I) Complexes with Spectacular Quantum Yields. J. Am. Chem. Soc. 2013, 135, 4712-4715.
24. Jones, G. C. H.; Jones, P. G.; Maddock, A. G.; Mays, M. J.; Vergnano, P. A.; Williams, A. F., Structure and bonding in gold(I) compounds. Part 3. Mossbauer spectra of three-co-ordinate complexes. J. Chem. Soc., Dalton Trans. 1977, (15), 1440-1443.
25. Usón, R.; Laguna, A.; Navarro, A.; Parish, R. V.; Moore, L. S., Synthesis and reactivity of perchlorate bis(tetrahydrothiophen)gold(I). 197Au Mossbauer spectra of three-coordinate gold(I) complexes. Inorg. Chim. Acta 1986, 112, 205-208.
26. Navarro, M.; Toledo, A.; Joost, M.; Amgoune, A.; Mallet-Ladeira, S.; Bourissou, D., 7E Complexes of PAP and PAN chelated gold(i). Chemical Communications 2019, 55, 7974-7977.
27. Yang, Y.; Eberle, L.; Mulks, F. F.; Wunsch, J. F.; Zimmer, M.; Rominger, F.; Rudolph, M.; Hashmi, A. S. K., Trans Influence of Ligands on the Oxidation of Gold(I) Complexes. J. Am. Chem. Soc. 2019, 141, 17414-17420.
28. Kleinhans, G.; Hansmann, M. M.; Guisado-Barrios, G.; Liles, D. C.; Bertrand, G.; Bezuidenhout, D. I., Nucleophilic T-Shaped (LXL)Au(I)-Pincer Complexes: Protonation and Alkylation. J. Am. Chem. Soc. 2016, /38, 15873-15876.
29. Luong, L. M. C.; Aristov, M. M.; Adams, A. V.; Walters, D. T.; Berry, J. F.; Olmstead, M. M.; Balch, A. L., Unsymmetrical Coordination of Bipyridine in Three-Coordinate Gold(I) Complexes. Inorg. Chem. 2020, 59, 4109-4117.
30. López-de-Luzuriaga, J. M.; Monge, M.; Olmos, M. E.; Rodriguez-Castillo, M.; Soldevilla, I.; Sundholm, D.; Valiev, R. R., Perhalophenyl Three-Coordinate Gold(I) Complexes as TADF Emitters: A Photophysical Study from Experimental and Computational Viewpoints. Inorg. Chem. 2020, 59, 14236-14244.
31. Joost, M.; Zeineddine, A.; Estevez, L.; Mallet—Ladeira, S.; Miqueu, K.; Amgoune, A.; Bourissou, D., Facile Oxidative Addition of Aryl Iodides to Gold(I) by Ligand Design: Bending Turns on Reactivity. J. Am. Chem. Soc. 2014, 136, 14654-14657.
32. Zeineddine, A.; Rekhroukh, F.; Sosa Carrizo, E. D.; Mallet-Ladeira, S.; Miqueu, K.; Amgoune, A.; Bourissou, D., Isolation of a Reactive Tricoordinate a-Oxo Gold Carbene Complex. Angew. Chem. Int. Ed. 2018, 57, 1306-1310.
33. McKeage, M. J.; Berners-Price, S. J.; Galettis, P.; Bowen, R. J.; Brouwer, W.; Ding, L.; Zhuang, L.; Baguley, B. C., Role of lipophilicity in determining cellular uptake and antitumour activity of gold phosphine complexes. Cancer Chemo. Pharm. 2000, 46, 343-350.
34. Mahepal, S.; Bowen, R.; Mamo, M. A.; Layh, M.; Jansen van Rensburg, C. E., The In Vitro Antitumour Activity of Novel, Mitochondrial-Interactive, Gold-Based Lipophilic Cations. Metal-Based Drugs 2008, 2008, 864653.
35. Vela, L.; Contel, M.; Palomera, L.; Azaceta, G.; Marzo, I., Iminophosphorane-organogold(III) complexes induce cell death through mitochondrial ROS production. J. Inorg. Biochem. 2011, 105, 1306-1313.
36. Barnard, P. J.; Berners-Price, S. J., Targeting the mitochondrial cell death pathway with gold compounds. Coord. Chem. Rev. 2007, 251, 1889-1902.
37. Hickey, J. L.; Ruhayel, R. A.; Barnard, P. J.; Baker, M. V.; Berners-Price, S. J.; Filipovska, A., Mitochondria-Targeted Chemotherapeutics: The Rational Design of Gold(I) N-Heterocyclic Carbene Complexes That Are Selectively Toxic to Cancer Cells and Target Protein Selenols in Preference to Thiols. J. Am. Chem. Soc. 2008, 130, 12570-12571.
38. Baker, M. V.; Barnard, P. J.; Berners-Price, S. J.; Brayshaw, S. K.; Hickey, J. L.; Skelton, B. W.; White, A. H., Cationic, linear Au(i) N-heterocyclic carbene complexes: synthesis, structure and anti-mitochondrial activity. Dalton Trans. 2006, (30), 3708-3715.
39. Che, C.-M.; Sun, R. W.-Y., Therapeutic applications of gold complexes: lipophilic gold(iii) cations and gold(i) complexes for anti-cancer treatment. Chem. Commun. 2011, 47, 9554-9560.
40. Zhang, C.; Fortin, P.-Y.; Barnoin, G.; Qin, X.; Wang, X.; Fernandez Alvarez, A.; Bijani, C.; Maddelein, M.-L.; Hemmert, C.; Cuvillier, 0.; Gornitzka, H., An Artemisinin-Derivative—(NHC)Gold(I) Hybrid with Enhanced Cytotoxicity through Inhibition of NRF2 Transcriptional Activity. Angew. Chem. Int. Ed. 2020, 59, 12062-12068.
41. Mertens, R. T.; Kim, J. H.; Jennings, W. C.; Parkin, S.; Awuah, S. G., Revisiting the reactivity of tetrachloroauric acid with N,N-bidentate ligands: structural and spectroscopic insights. Dalton Trans. 2019, 48, 2093-2099.
42. Johnstone, T. C.; Park, G. Y.; Lippard, S. J., Understanding and improving platinum anticancer drugs--phenanthriplatin. Anticancer Res. 2014, 34, 471-476.
43. Shen, D.-W.; Pouliot, L. M.; Hall, M. D.; Gottesman, M. M., Cisplatin resistance: a cellular self-defense mechanism resulting from multiple epigenetic and genetic changes. Pharmacol. Rev. 2012, 64, 706-721.
44. Damia, G.; Broggini, M., Platinum Resistance in Ovarian Cancer: Role of DNA Repair. Cancers (Basel) 2019, 11, 119.
45. Mylavarapu, S.; Das, A.; Roy, M., Role of BRCA Mutations in the Modulation of Response to Platinum Therapy. Front. Oncol. 2018, 8, 16-16.
46. Wang, F.-Y.; Tang, X.-M.; Wang, X.; Huang, K.-B.; Feng, H.-W.; Chen, Z.-F.; Liu, Y.-N.; Liang, H., Mitochondria-targeted platinum(II) complexes induce apoptosis-dependent autophagic cell death mediated by ER-stress in A549 cancer cells. Eur. J. Med. Chem. 2018, 155, 639-650.
47. Andrea, E., Mitochondria-Targeting Anticancer Metal Complexes. Curr. Med. Chem. 2019, 26, 694-728.
48. Tong, K.-C.; Lok, C.-N.; Wan, P.-K.; Hu, D.; Fung, Y. M. E.; Chang, X.-Y.; Huang, S.; Jiang, H.; Che, C.-M., An anticancer gold(III)-activated porphyrin scaffold that covalently modifies protein cysteine thiols. Proc. Natl. Acad. Sci. 2020, 117, 1321-1329.
49. Dixon, Scott J.; Lemberg, Kathryn M.; Lamprecht, Michael R.; Skouta, R.; Zaitsev, Eleina M.; Gleason, Caroline E.; Patel, Darpan N.; Bauer, Andras J.; Cantley, Alexandra M.; Yang, Wan S.; Morrison, B.; Stockwell, Brent R., Ferroptosis: An Iron-Dependent Form of Nonapoptotic Cell Death. Cell 2012, 149, 1060-1072.
50. Wasilewski, M.; Scorrano, L., The changing shape of mitochondrial apoptosis. Trends Endocrinol. Metab. 2009, 20, 287-94.
51. Schleiff, E.; Turnbull, J. L., Functional and Structural Properties of the Mitochondrial Outer Membrane Receptor Tom20. Biochemistry 1998, 37, 13043-13051.
52. Qi, Y.; Yan, L.; Yu, C.; Guo, X.; Zhou, X.; Hu, X.; Huang, X.; Rao, Z.; Lou, Z.; Hu, J., Structures of human mitofusin 1 provide insight into mitochondrial tethering. J. Cell Biol. 2016, 215, 621-629.
53. Chen , H.; Detmer , S. A.; Ewald , A. J.; Griffin , E. E.; Fraser , S. E.; Chan , D. C., Mitofusins Mfn1 and Mfn2 coordinately regulate mitochondrial fusion and are essential for embryonic development. J. Cell Biol. 2003, 160, 189-200.
54. Gandre-Babbe, S.; Bliek, A. M. v. d., The Novel Tail-anchored Membrane Protein Mff Controls Mitochondrial and Peroxisomal Fission in Mammalian Cells. Mol. Biol. Cell 2008, 19, 2402-2412.
55. Olichon, A.; Baricault, L.; Gas, N.; Guillou, E.; Valette, A.; Belenguer, P.; Lenaers, G., Loss of OPA1 Perturbates the Mitochondrial Inner Membrane Structure and Integrity, Leading to Cytochrome c Release and Apoptosis. J. Biol. Chem. 2003, 278, 7743-7746.
56. Milacic, V.; Chen, D.; Ronconi, L.; Landis-Piwowar, K. R.; Fregona, D.; Dou, Q. P., A Novel Anticancer Gold(III) Dithiocarbamate Compound Inhibits the Activity of a Purified 20S Proteasome and 26S Proteasome in Human Breast Cancer Cell Cultures and Xenografts. Cancer Res. 2006, 66, 10478.
57. Vela, L.; Contel, M.; Palomera, L.; Azaceta, G.; Marzo, I., Iminophosphorane-organogold(III) complexes induce cell death through mitochondrial ROS production. J. Biochem. 2011, 105, 1306-1313.
58. Marmol, I.; Virumbrales-Muñoz, M.; Quero, J.; Sánchez-de-Diego, C.; Fernandez, L.; Ochoa, I.; Cerrada, E.; Yoldi, M. J. R., Alkynyl gold(I) complex triggers necroptosis via ROS generation in colorectal carcinoma cells. J. Inorg. Biochem. 2017, 176, 123-133.
59. Zhang, C.; Maddelein, M.-L.; Wai-Yin Sun, R.; Gornitzka, H.; Cuvillier, O.; Hemmert, C., Pharmacomodulation on Gold-NHC complexes for anticancer applications-is lipophilicity the key point? Eur. J. Med. Chem. 2018, 157, 320-332.
60. Ganga Reddy, V.; Srinivasa Reddy, T.; Privér, S. H.; Bai, Y.; Mishra, S.; Wlodkowic, D.; Mirzadeh, N.; Bhargava, S., Synthesis of Gold(I) Complexes Containing Cinnamide: In Vitro Evaluation of Anticancer Activity in 2D and 3D Spheroidal Models of Melanoma and In Vivo Angiogenesis. Inorg. Chem. 2019, 58, 5988-5999.
61. Molina, J. R.; Sun, Y.; Protopopova, M.; Gera, S.; Bandi, M.; Bristow, C.; McAfoos, T.; Morlacchi, P.; Ackroyd, J.; Agip, A.-N. A.; Al-Atrash, G.; Asara, J.; Bardenhagen, J.; Carrillo, C. C.; Carroll, C.; Chang, E.; Ciurea, S.; Cross, J. B.; Czako, B.; Deem, A.; Daver, N.; de Groot, J. F.; Dong, J.-W.; Feng, N.; Gao, G.; Gay, J.; Do, M. G.; Greer, J.; Giuliani, V.; Han, J.; Han, L.; Henry, V. K.; Hirst, J.; Huang, S.; Jiang, Y.; Kang, Z.; Khor, T.; Konoplev, S.; Lin, Y.-H.; Liu, G.; Lodi, A.; Lofton, T.; Ma, H.; Mahendra, M.; Matre, P.; Mullinax, R.; Peoples, M.; Petrocchi, A.; Rodriguez-Canale, J.; Serreli, R.; Shi, T.; Smith, M.; Tabe, Y.; Theroff, J.; Tiziani, S.; Xu, Q.; Zhang, Q.; Muller, F.; DePinho, R. A.; Toniatti, C.; Draetta, G. F.; Heffernan, T. P.; Konopleva, M.; Jones, P.; Di Francesco, M. E.; Marszalek, J. R., An inhibitor of oxidative phosphorylation exploits cancer vulnerability. Nat. Med. 2018, 24, 1036-1046.
62. Shi, Y.; Lim, S. K.; Liang, Q.; Iyer, S. V.; Wang, H.-Y.; Wang, Z.; Xie, X.; Sun, D.; Chen, Y.-J.; Tabar, V.; Gutin, P.; Williams, N.; De Brabander, J. K.; Parada, L. F., Gboxin is an oxidative phosphorylation inhibitor that targets glioblastoma. Nature 2019, 567, 341-346.
63. Perelman, A.; Wachtel, C.; Cohen, M.; Haupt, S.; Shapiro, H.; Tzur, A., JC-1: alternative excitation wavelengths facilitate mitochondrial membrane potential cytometry. Cell Death & Disease 2012, 3, e430-e430.
64. Holmström, K. M.; Kostov, R. V.; Dinkova-Kostova, A. T., The multifaceted role of Nrf2 in mitochondrial function. Curr Opin Toxicol 2016, 1, 80-91.
65. Mirabelli, C. K.; Johnson, R. K.; Sung, C. M.; Faucette, L.; Muirhead, K.; Crooke, S. T., Evaluation of the in vivo antitumor activity and in vitro cytotoxic properties of auranofin, a coordinated gold compound, in murine tumor models. Cancer Res. 1985, 45, 32-9.
66. Fernández-Gallardo, J.; Elie, B. T.; Sadhukha, T.; Prabha, S.; Sandú, M.; Rotenberg, S. A.; Ramos, J. W.; Contel, M., Heterometallic titanium—gold complexes inhibit renal cancer cells in vitro and in vivo. Chem. Sci. 2015, 6, 5269-5283.
67. Zou, T.; Lum, C. T.; Lok, C.-N.; Zhang, J.-J.; Che, C.-M., Chemical biology of anticancer gold(iii) and gold(i) complexes. Chem. Soc. Rev. 2015, 44, 8786-8801.
68. Wolfgang, W.; Oyinlola, D.; Cillian, O. B.; Ingo, O.; Goar, S.-S.; Claudia, S.; Carsta, W.; Xiangming, Z.; Matthias, T., In Vitro and In Vivo Investigations into the Carbene Gold Chloride and Thioglucoside Anticancer Drug Candidates NHC-AuCl and NHC-AuSR. Lett. Drug Design Disc. 2017, 14, 125-134.
69. Ramsay, W. J.; Bell, N. A. W.; Qing, Y.; Bayley, H., Single-Molecule Observation of the Intermediates in a Catalytic Cycle. J. Am. Chem. Soc. 2018, 140, 17538-17546.
70. Su, H.; Wang, Y.; Ren, L.; Yuan, P.; Teo, B. K.; Lin, S.; Zheng, L.; Zheng, N., Fractal Patterns in Nucleation and Growth of Icosahedral Core of [AunAg4-n(SC6H3F2)30]4— (n=0-12) via ab Initio Synthesis: Continuously Tunable Composition Control. Inorg. Chem. 2019, 58, 259-264.
71. Do, T. G.; Hupf, E.; Lork, E.; Kogel, J. F.; Mohr, F.; Brown, A.; Toyoda, R.; Sakamoto, R.; Nishihara, H.; Mebs, S.; Beckmann, J., Aurophilicity and Photoluminescence of (6-Diphenylpnicogenoacenaphth-5-yl)gold Compounds. Eur. J. Inorg. Chem. 2019, 2019, 647-659.
72. Dean, T. C.; Yang, M.; Liu, M.; Grayson, J. M.; DeMartino, A. W.; Day, C. S.; Lee, J.; Furdui, C. M.; Bierbach, U., Human Serum Albumin-Delivered [Au(PEt3)]+Is a Potent Inhibitor of T Cell Proliferation. ACS Med. Chem. Lett. 2017, 8, 572-576.
73. Parkin, S.; Hope, H., Macromolecular Cryocrystallography: Cooling, Mounting, Storage and Transportation of Crystals. J. Appl. Crystallog. 1998, 31, 945-953.
74. Hope, H., X-RAY CRYSTALLOGRAPHY-A FAST, FIRST-RESORT ANALYTICAL TOOL. Prog. Inorg. Chem., Vol 41 1994, 41, 1-19.
75. Bruker, “APEX2” Bruker-AXS. Madison, WI. USA, 2006.
76. Krause, L.; Herbst-Irmer, R.; Sheldrick, G. M.; Stalke, D., Comparison of silver and molybdenum microfocus X-ray sources for single-crystal structure determination. J. Appl. Crystallogr. 2015, 48, 3-10.
77. Sheldrick, G. M., SADABS, Program for bruker area detector absorption correction. University of Gottingen, Gottingen, 1997.
78. Sheldrick, G. M., Crystal structure refinement with SHELXL. Acta Crystallogr. C Struct. Chem. 2015, 71, 3-8.
79. Sheldrick, G. M., SHELXT- ntegrated space-group and crystal-structure determination. Acta Crystallogr. A Found. Adv. 2015, 71, 3-8.
80. Sheldrick, G., A short history of SHELX. Acta Crystallogr. Sec. A 2008, 64, 112-122.
81. Spek, A. L., Structure validation in chemical crystallography. Acta Crystallogr. D Biol. Crystallogr. 2009, 65 (Pt 2), 148-55.
82. Parkin, S., Expansion of scalar validation criteria to three dimensions: the R tensor. Erratum. Acta Crystallogr. Sec. A 2000, 56, 317.
83. van Oosterwijk, J. G.; Li, C.; Yang, X.; Opferman, J. T.; Sherr, C. J., Small mitochondrial Arf (smArf) protein corrects p53-independent developmental defects of <em>Arf</em> tumor suppressor-deficient mice. P.N.A.S. 2017, 114, 7420-7425.
84. Wang, G.; McCain, M. L.; Yang, L.; He, A.; Pasqualini, F. S.; Agarwal, A.; Yuan, H.; Jiang, D.; Zhang, D.; Zangi, L.; Geva, J.; Roberts, A. E.; Ma, Q.; Ding, J.; Chen, J.; Wang, D.-Z.; Li, K.; Wang, J.; Wanders, R. J. A.; Kulik, W.; Vaz, F. M.; Laflamme, M. A.; Murry, C. E.; Chien, K. R.; Kelley, R. I.; Church, G. M.; Parker, K. K.; Pu, W. T., Modeling the mitochondrial cardiomyopathy of Barth syndrome with induced pluripotent stem cell and heart-on-chip technologies. Nat. Med. 2014, 20, 616-623.

It will be understood that various details of the presently disclosed subject matter can be changed without departing from the scope of the subject matter disclosed herein. Furthermore, the foregoing description is for the purpose of illustration only, and not for the purpose of limitation.

1. A compound of the formula: 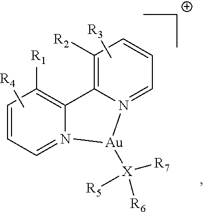
or a pharmaceutically-acceptable salt thereof,
wherein
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl;
R₅ and R₆ are each 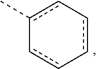
or when X is C then R₅ and R₆ taken together with the C to which they are bound can form a 5-membered ring that is substituted or unsubstituted;
R₇ is H or 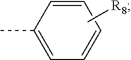
and
R₈ is selected from the group consisting of H, alkyl, cycloalkyl, aryl, and heterocycloalkyl.

2. The compound of claim 1, of the formula: 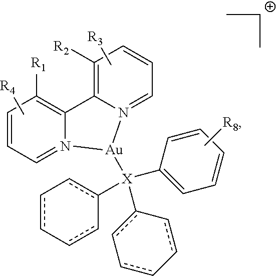
or a pharmaceutically-acceptable salt thereof,
wherein
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

3. The compound of claim 1, of the formula: 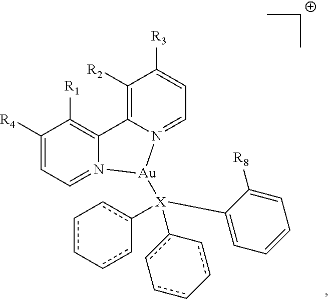
or a pharmaceutically-acceptable salt thereof,
wherein
X is C, P or As;
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

4. The compound of claim 1, of the formula: 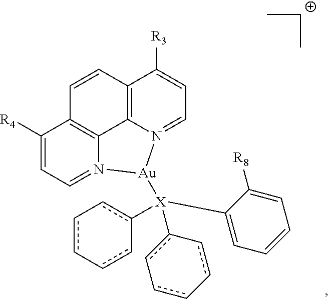
or a pharmaceutically-acceptable salt thereof,
wherein
X is C, P or As;
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

5. The compound of claim 1, of the formula: 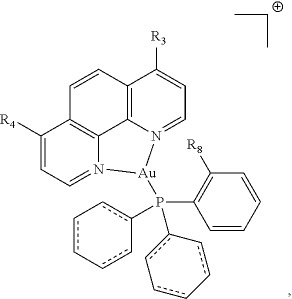
or a pharmaceutically-acceptable salt thereof,
wherein
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

6. The compound of claim 1, of the formula: 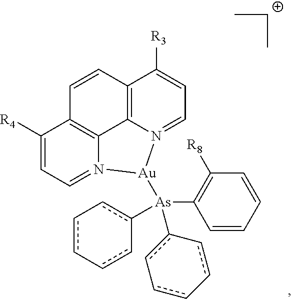
or a pharmaceutically-acceptable salt thereof,
wherein
R₃ and R₄ are independently selected from the group consisting of H, alkyl, cycloalkyl, aryl, and heterocycloalkyl; and
R₈ is selected from the group consisting of H, alkyl, cycloalkyl, aryl, and heterocycloalkyl.

7. The compound of claim 1, of the formula: 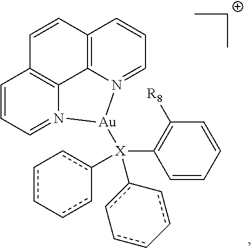
or a pharmaceutically-acceptable salt thereof, wherein R₈ is selected from the group consisting of H, alkyl, cycloalkyl, aryl, and heterocycloalkyl.

8. The compound of claim 1, of the formula: 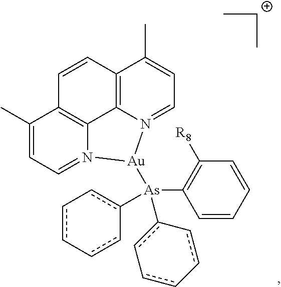
or a pharmaceutically-acceptable salt thereof, wherein R₈ is selected from the group consisting of H, alkyl, cycloalkyl, aryl, and heterocycloalkyl.

9. The compound of claim 1, of the formula: 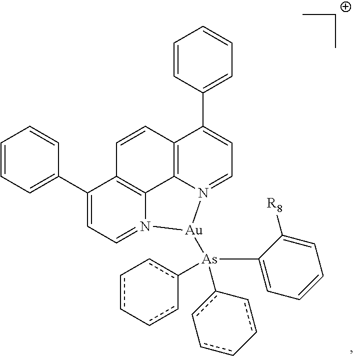
or a pharmaceutically-acceptable salt thereof,
wherein R₈ is selected from the group consisting of H, alkyl, cycloalkyl, aryl, and heterocycloalkyl.

10. The compound of claim 1, of the formula: 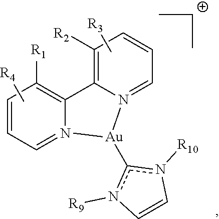
or a pharmaceutically-acceptable salt thereof,
wherein
R₁ and R₂ are independently selected from the group consisting of H, alkyl, cycloalkyl, aryl, and heterocycloalkyl, or R₁ and R₂, taken together with the carbons to which they are bound, form a 5-7-membered ring;
R₃ and R₄ are independently selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

11. The compound of claim 1, of the formula: 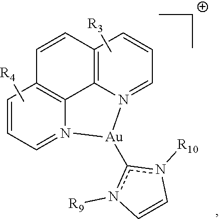
or a pharmaceutically-acceptable salt thereof,
wherein
R₃ and R₄ are independently selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

12. The compound of claim 1, of the formula: 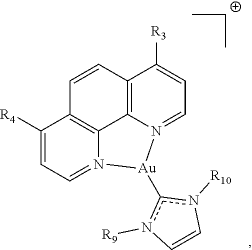
or a pharmaceutically-acceptable salt thereof,
wherein
R₃ and R₄ are independently selected from the group consisting of H, alkyl, cycloalkyl, aryl, and heterocycloalkyl; and
R₉ and R₁₀ are each independently selected from the group consisting of H, alkyl, cycloalkyl, aryl, and heterocycloalkyl.

13. The compound of claim 1, of a formula selected from the group consisting of: 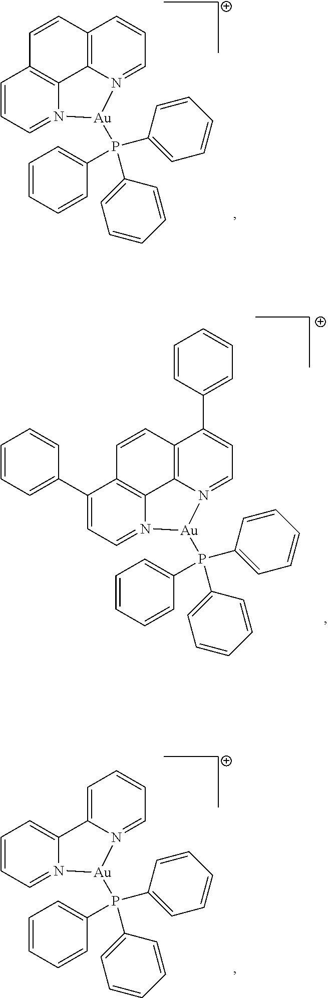 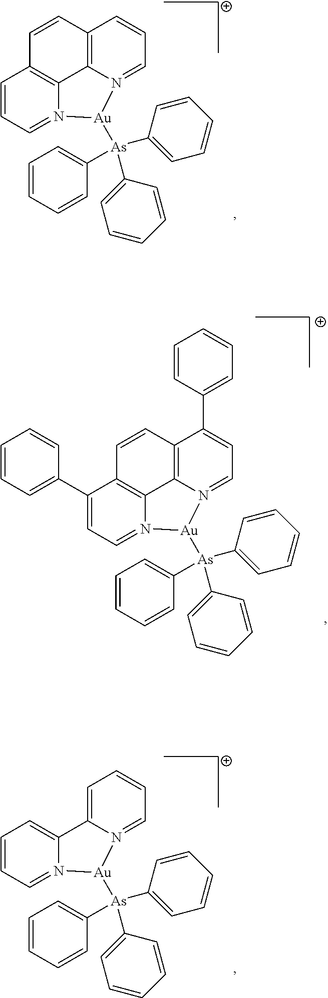 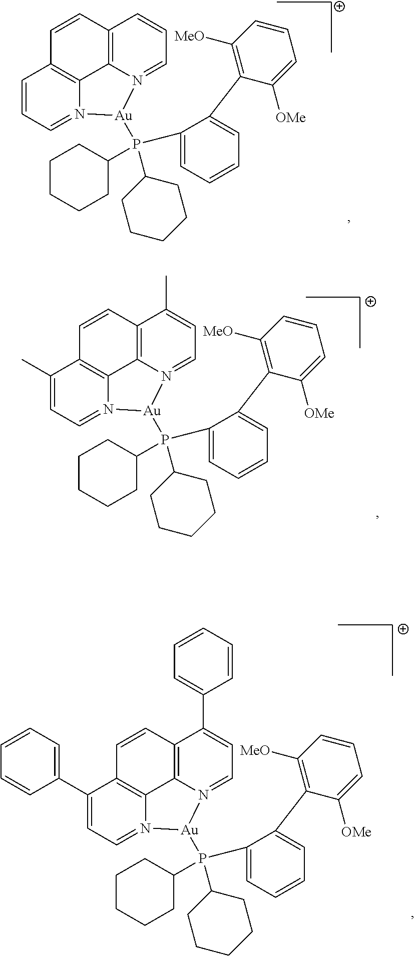 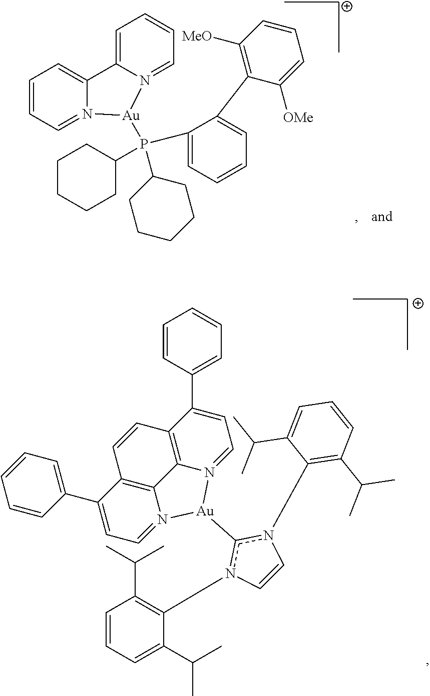
or a pharmaceutically-acceptable salt thereof

14. A pharmaceutical composition comprising the compound of claim 1 and a pharmaceutically-acceptable carrier.

15. A method of conferring anti-cancer activity to a cancer cell, comprising: contacting a cancer cell with an effective amount of the compound of claim 1.

16. The method of claim 15, wherein the conferring anti-cancer activity results in one or more of inhibiting proliferation of the cancer cell, inhibiting metastasis, and killing the cancer cell.

17-20. (canceled)

21. A method of modulating mitochondrial function in a cell, comprising: contacting a cell with an effective amount of the compound of claim 1.

22-26. (canceled)

27. A method of increasing reactive oxygen species (ROS) in a cell, comprising: contacting a cell with an effective amount of the compound of claim 1.

28. The method of claim 27, wherein the effective amount is from about 10 nM to about 100 μM.

29. The method of claim 27, wherein the cell is a cancer cell.

30-33. (canceled)
